SEQUENCE LISTING (30 sequences)

SEQ ID NO: 1 - DNA, 110 bp, synthetic construct
  source 1..110: mol_type = other DNA
tcgtggatctgtctctgctctgtttatggcccttcggtaattcactgactgagactgttcacagtgaattctaccagtgccatacacagaacaggagtcacactctgcgg

SEQ ID NO: 2 - RNA, 110 nt, Homo sapiens
  source 1..110: mol_type = transcribed RNA
ccgcagagtgtgactcctgttctgtgtatggcactggtagaattcactgtgaacagtctcagtcagtgaattaccgaagggccataaacagagcagagacagatccacga

SEQ ID NO: 3 - RNA, 22 nt, Homo sapiens
  source 1..22: mol_type = transcribed RNA
tatggcactggtagaattcact

SEQ ID NO: 4 - RNA, 22 nt, Homo sapiens
  source 1..22: mol_type = transcribed RNA
gtgaattaccgaagggccataa

SEQ ID NO: 5 - DNA, 22 bp, synthetic construct
  source 1..22: mol_type = other DNA
agtgaattctaccagtgccata

SEQ ID NO: 6 - DNA, 22 bp, synthetic construct
  source 1..22: mol_type = other DNA
ttatggcccttcggtaattcac

SEQ ID NO: 7 - DNA, 330 bp, synthetic construct
  source 1..330: mol_type = other DNA
tcgtggatctgtctctgctctgtttatggcccttcggtaattcactgactgagactgttcacagtgaattctaccagtgccatacacagaacaggagtcacactctgcggtcgtggatctgtctctgctctgtttatggcccttcggtaattcactgactgagactgttcacagtgaattctaccagtgccatacacagaacaggagtcacactctgcggtcgtggatctgtctctgctctgtttatggcccttcggtaattcactgactgagactgttcacagtgaattctaccagtgccatacacagaacaggagtcacactctgcgg

SEQ ID NO: 8 - DNA, 102 bp, synthetic construct
  source 1..102: mol_type = other DNA
agtgaattctaccagtgccataataactaaaagattcggaagtgaattctaccagtgccataataactaaaagattcggaagtgaattctaccagtgccata

SEQ ID NO: 9 - DNA, 222 bp, synthetic construct
  source 1..222: mol_type = other DNA
agtgaattctaccagtgccataataactaaaagattcggattatggcccttcggtaattcacataactaaaagattcggaagtgaattctaccagtgccataataactaaaagattcggattatggcccttcggtaattcacataactaaaagattcggaagtgaattctaccagtgccataataactaaaagattcggattatggcccttcggtaattcac

SEQ ID NO: 10 - DNA, 24 bp, synthetic construct
  source 1..24: mol_type = other DNA
agtgtgagttctaccattgccaaa

SEQ ID NO: 11 - RNA, 24 nt, Homo sapiens
  source 1..24: mol_type = transcribed RNA
tttggcaatggtagaactcacact

SEQ ID NO: 12 - DNA, 21 bp, synthetic construct
  source 1..21: mol_type = other DNA
tagttggcaagtctagaacca

SEQ ID NO: 13 - RNA, 21 nt, Homo sapiens
  source 1..21: mol_type = transcribed RNA
tggttctagacttgccaacta

SEQ ID NO: 14 - DNA, 18 bp, synthetic construct
  source 1..18: mol_type = other DNA
ataactaaaagattcgga

SEQ ID NO: 15 - DNA, 18 bp, synthetic construct
  source 1..18: mol_type = other DNA
aatatatatatattatta

SEQ ID NO: 16 - DNA, 18 bp, synthetic construct
  source 1..18: mol_type = other DNA
aaaaacatataaaataat

SEQ ID NO: 17 - DNA, 18 bp, synthetic construct
  source 1..18: mol_type = other DNA
ctttcttttcccaatttt

SEQ ID NO: 18 - DNA, 19 bp, synthetic construct
  source 1..19: mol_type = other DNA
gagcgcaccatcttcttca

SEQ ID NO: 19 - DNA, 18 bp, synthetic construct
  source 1..18: mol_type = other DNA
ttcagctcgatgcggttc

SEQ ID NO: 20 - DNA, 23 bp, synthetic construct
  source 1..23: mol_type = other DNA
aggacgacggcaactacaagacc

SEQ ID NO: 21 - DNA, 110 bp, synthetic construct
  source 1..110: mol_type = other DNA
gtgccggctgagtcctcgccccatagttggcaagtctagaaccaccggatcctgttacctcaccagtgtgagttctaccattgccaaaaacggggggaggcaagcagctc

SEQ ID NO: 22 - RNA, 110 nt, Homo sapiens
  source 1..110: mol_type = transcribed RNA
gagctgcttgcctccccccgtttttggcaatggtagaactcacactggtgaggtaacaggatccggtggttctagacttgccaactatggggcgaggactcagccggcac

SEQ ID NO: 23 - DNA, 330 bp, synthetic construct
  source 1..330: mol_type = other DNA
gtgccggctgagtcctcgccccatagttggcaagtctagaaccaccggatcctgttacctcaccagtgtgagttctaccattgccaaaaacggggggaggcaagcagctcgtgccggctgagtcctcgccccatagttggcaagtctagaaccaccggatcctgttacctcaccagtgtgagttctaccattgccaaaaacggggggaggcaagcagctcgtgccggctgagtcctcgccccatagttggcaagtctagaaccaccggatcctgttacctcaccagtgtgagttctaccattgccaaaaacggggggaggcaagcagctc

SEQ ID NO: 24 - DNA, 78 bp, synthetic construct
  source 1..78: mol_type = other DNA
tttcccatattggcactgcacatgattgctcagagcggagagacacaagcaaaaatgtgctagtgccaaaatcggcca

SEQ ID NO: 25 - RNA, 78 nt, Homo sapiens
  source 1..78: mol_type = transcribed RNA
tggccgattttggcactagcacatttttgcttgtgtctctccgctctgagcaatcatgtgcagtgccaatatgggaaa

SEQ ID NO: 26 - DNA, 234 bp, synthetic construct
  source 1..234: mol_type = other DNA
tttcccatattggcactgcacatgattgctcagagcggagagacacaagcaaaaatgtgctagtgccaaaatcggccatttcccatattggcactgcacatgattgctcagagcggagagacacaagcaaaaatgtgctagtgccaaaatcggccatttcccatattggcactgcacatgattgctcagagcggagagacacaagcaaaaatgtgctagtgccaaaatcggcca

SEQ ID NO: 27 - DNA, 23 bp, synthetic construct
  source 1..23: mol_type = other DNA
agcaaaaatgtgctagtgccaaa

SEQ ID NO: 28 - RNA, 23 nt, Homo sapiens
  source 1..23: mol_type = transcribed RNA
tttggcactagcacatttttgct

SEQ ID NO: 29 - DNA, 22 bp, synthetic construct
  source 1..22: mol_type = other DNA
catattggcactgcacatgatt

SEQ ID NO: 30 - RNA, 22 nt, Homo sapiens
  source 1..22: mol_type = transcribed RNA
aatcatgtgcagtgccaatatg

(19) United States
(12) Patent Application Publication
(10) Pub. No.: US 2026/0009028 A1
(43) Pub. Date: Jan. 8, 2026

(54) REGULATORY SEQUENCES COMPRISING MicroRNA TARGET SITES

(71) Applicant: SENSORION, Montpellier (FR)

(72) Inventors: Laurent DÉSIRÉ, Montpellier (FR); Patrice VIDAL, Montpellier (FR); Arnaud GIESE, Montpellier (FR); Rafik BOUDRA, Montpellier (FR)

(73) Assignee: SENSORION, Montpellier (FR)

(21) Appl. No.: 18/992,231

(22) PCT Filed: Jul. 7, 2023
(86) PCT No.: PCT/EP2023/068912

§ 371 Date: Jan. 8, 2025

(30) Foreign Application Priority Data
Jul. 8, 2022 (EP) 22306028.6

(51) Int. Cl. C12N 15/113; C12N 15/86

(52) U.S. Cl. CPC C12N 15/113; C12N 15/86; C12N 2750/14143

(57) ABSTRACT

An isolated nucleic acid sequence including at least two copies of a miRNA target site of the miR183 family having a sequence as set forth in SEQ ID NO: 1, SEQ ID NO: 21, or SEQ ID NO: 24, or a sequence having at least 90% identity with any one of SEQ ID NO: 1, SEQ ID NO: 21, or SEQ ID NO: 24. Also a vector including a regulatory element having at least one copy of this miRNA target site of the miR183 family. The isolated nucleic acid sequence and the vector may be particularly useful for controlling the expression of a gene of interest, for example when designing and developing gene therapies.

### FIELD OF INVENTION

The present invention relates to regulatory sequences comprising microRNAs target sites, in particular a miRNA target site of the miR183 family, and their use for controlling the expression of a nucleic acid sequence of interest, such as a gene of interest.

### BACKGROUND OF INVENTION

MicroRNAs (miRNAs or miRs) are an abundant class of short, naturally occurring, non-coding RNAs of about 20 nucleotides in length that play important roles in regulating gene expression. The majority of miRNAs are transcribed from DNA sequences by RNA polymerases II or III into large RNA precursors called primary miRNAs (or pri-miRNAs). The pri-miRNAs are processed into hairpin precursor miRNAs (or pre-miRNAs) of about 70 to about 120 nucleotides in length, which are themselves further processed into mature miRNAs of about 20 nucleotides in length. In most cases, after recognizing their target sites, generally located in 3′-untranslated transcribed regions (UTRs), mature miRNAs achieve complete or incomplete base complementary pairing, which leads to mRNA cleavage and inhibition of mRNA translation into protein. miRNAs thus play an important role in regulating post-transcriptional gene expression in a sequence specific manner.

Accordingly, strategies for selective gene silencing based on the use of vectors comprising one or several copies of a miRNA target site operably linked to or inserted in the nucleic acid sequence of a gene of interest have been developed and described in the art. In these strategies, the miRNA target site consists in a short nucleotide sequence complementary to a specific mature miRNA. Upon introduction of the vector into cells expressing the miRNA, which specifically recognizes the miRNA target site, the miRNA suppresses the expression of the gene of interest in said cells through its interaction with the miRNA target site.

Thousands of miRNAs have been discovered in many organisms, with more than 2000 mature miRNAs identified in Homo sapiens. miRNAs can be grouped into clusters, defined as several miR genes located adjacent to each other on a chromosome, which are transcribed as one long primary miRNA and then processed into individual hairpin precursor miRNAs. Furthermore, high sequence homology between the miRNAs of a cluster classifies them as a family and permits both common and unique mRNA targets for the miRNAs of the family. Among the identified miRNA families, the miR183 family consists of 3 homologous miRNA: miR183, miR96 and miR182. The miRNAs of the miR183 family are highly expressed in sensory cells in the eye, nose and inner ear, and are essential for their development.

A number of sensory impairments, in particular blindness and deafness, are of genetic origin, that is to say are caused by the presence of a mutation in a gene. Such sensory impairments are usually termed hereditary or congenital sensory impairments. Currently, a number of gene therapy approaches are being developed with the aim of treating sensory impairments caused by a single gene mutation. Treatments based on gene therapy rely on providing an intact gene to compensate for the defective gene responsible for the congenital sensory impairment. Of note, it is of utmost importance that the gene introduced through gene therapy only be specifically expressed in the cells and tissues where expression of said gene naturally occurs. In particular, it is critical to ensure that the gene introduced through gene therapy is not expressed in cells and tissues where expression of the gene could induce any deleterious effects. Thus, there is a need for regulatory sequences and vectors allowing to control the expression of a gene of interest. In particular, there is a need for regulatory sequences and vectors allowing to prevent or repress the expression of a gene of interest in cells and tissues where said expression could induce any deleterious effects.

The Inventors surprisingly demonstrated that the introduction of a vector comprising a gene and at least one copy of a “precursor miR183 target site” into cells expressing miR183 significantly repressed expression of the gene in said cells. As used herein, the so-called “precursor miR183 target site” refers to a miR183 target site having a sequence complementary to the human sequence of the precursor miR183. A similar regulatory effect was observed with other “precursor miRNA target sites” of the miR183 family, i.e., the so-called “precursor miR182 target site” referring to a miR182 target site having a sequence complementary to the human sequence of the precursor miR182, and the so-called “precursor miR96 target site” referring to a miR96 target site having a sequence complementary to the human sequence of the precursor miR96. Strikingly, the so-called precursor miR183 target site was significantly more efficient in repressing the gene expression in the cells expressing miR183 than a shorter miR183 target site having a sequence complementary to the human sequence of the mature miR183. The Inventors also demonstrated that the injection into mice inner ear of a vector comprising a gene and at least one copy of the so-called precursor miR183 target site significantly repressed expression of the gene in inner hair cells and outer hair cells of the cochlea.

These results thus establish that vectors comprising at least one copy of a so-called precursor miRNA target site of the miR183 family could be useful tools for selectively and effectively repressing unwanted gene expression in sensory cells. Such vectors could be of particular interest in gene therapy approaches seeking to treat congenital sensory impairments.

### SUMMARY

The present invention thus relates to an isolated nucleic acid sequence comprising at least two copies of a miRNA target site of the miR183 family, wherein said miRNA target site has a sequence as set forth in SEQ ID NO: 1, SEQ ID NO: 21, or SEQ ID NO: 24, or a sequence having at least 90% identity with any one of SEQ ID NO: 1, SEQ ID NO: 21, or SEQ ID NO: 24. In some embodiments, the present invention relates to an isolated nucleic acid sequence comprising at least two copies of a miR183 target site having a sequence as set forth in SEQ ID NO: 1 or a sequence having at least 90% identity with SEQ ID NO: 1. In some embodiment, the isolated nucleic acid sequence comprises two to six copies of the miRNA target site of the miR183 family, for example two to six copies of the miR183 target site having a sequence as set forth in SEQ ID NO: 1 or a sequence having at least 90% identity with SEQ ID NO: 1. In some embodiments, the isolated nucleic acid sequence comprises three copies of the miRNA target site of the miR183 family, for example three copies of the miR183 target site having a sequence as set forth in SEQ ID NO: 1 or a sequence having at least 90% identity with SEQ ID NO: 1. In some embodiments, the isolated nucleic acid sequence comprises a sequence as set forth in SEQ ID NO: 7, SEQ ID NO: 23, or SEQ ID NO: 26, or a sequence having at least 90% identity with any one of SEQ ID NO: 7, SEQ ID NO: 23, or SEQ ID NO: 26. In some embodiments, the isolated nucleic acid sequence comprises a sequence as set forth in SEQ ID NO: 7 or a sequence having at least 90% identity with SEQ ID NO: 7. In some embodiments, the copies of the miRNA target site of the miR183 family, for example the copies of the miR183 target site, are separated by a spacer.

The present invention also relates to an expression cassette comprising a promoter, a gene of interest, and a regulatory element comprising at least one copy of a miRNA target site of the miR183 family, wherein said miRNA target site has a sequence as set forth in SEQ ID NO: 1, SEQ ID NO: 21, or SEQ ID NO: 24, or a sequence having at least 90% identity with any one of SEQ ID NO: 1, SEQ ID NO: 21, or SEQ ID NO: 24. The present invention also relates to a vector comprising a regulatory element comprising at least one copy of a miRNA target site of the miR183 family, wherein said miRNA target site has a sequence as set forth in SEQ ID NO: 1, SEQ ID NO: 21, or SEQ ID NO: 24, or a sequence having at least 90% identity with any one of SEQ ID NO: 1, SEQ ID NO: 21, or SEQ ID NO: 2. In some embodiments, the regulatory element comprises at least one copy of a miR183 target site having a sequence as set forth in SEQ ID NO: 1 or a sequence having at least 90% identity with SEQ ID NO: 1. In some embodiments, the regulatory element comprises two to six copies, preferably three copies, of the miRNA target site of the miR183 family (for example of the miR183 target site having a sequence as set forth in SEQ ID NO: 1 or a sequence having at least 90% identity with SEQ ID NO: 1), the copies being optionally separated by a spacer. In some embodiments, the regulatory element comprises a sequence as set forth in SEQ ID NO: 7, SEQ ID NO: 23, or SEQ ID NO: 26, or a sequence having at least 90% identity with any one of SEQ ID NO: 7, SEQ ID NO: 23, or SEQ ID NO: 26. In some embodiments, the regulatory element comprises a sequence as set forth in SEQ ID NO: 7 or a sequence having at least 90% identity with SEQ ID NO: 7. In some embodiments, the regulatory element further comprises at least one copy of another miRNA target site, preferably at least one copy of another miRNA target site of the miR183 family. In some embodiments, the regulatory element comprises a miR183 target site having a sequence as set forth in SEQ ID NO: 1 or a sequence having at least 90% identity with SEQ ID NO: 1 and at least one copy of another miRNA target site, preferably at least one copy of another miRNA target site of the miR183 family, more preferably a miR182 target site.

In some embodiments, the regulatory element, comprised within an expression cassette as described herein or within a vector as described herein further comprising a gene of interest, is operably linked to or inserted in the gene of interest, preferably inserted in the 3′-UTR of the gene of interest.

The present invention also relates to a pharmaceutical composition comprising the isolated nucleic acid sequence as described herein, or the expression cassette as described herein, or the vector as described herein, and at least one pharmaceutically acceptable excipient.

The present invention also relates to the isolated nucleic acid sequence as described herein, or the expression cassette as described herein, or the vector as described herein, or the pharmaceutical composition as described herein, for use as a medicament.

The present invention also relates to the use of the isolated nucleic acid sequence as described herein, or of the expression cassette as described herein, or of the vector as described herein, for specifically expressing a gene of interest in cells not expressing a miRNA of the miR183 family, for example in cells not expressing miR183.

### Definitions

In the present invention, the following terms have the following meanings:

The terms “a” and “an” refer to one or to more than one (i.e., to at least one) of the grammatical object of the article. By way of example, “an element” means one element or more than one element.

“About” preceding a figure encompasses plus or minus 10%, or less, of the value of said figure. It is to be understood that the value to which the term “about” refers is itself also specifically, and preferably, disclosed.

“Encoding”, as in encoding sequence, refers to the inherent property of a specific sequence of nucleotides in a nucleic acid, such as a gene, a complementary DNA (cDNA), or a messenger RNA (mRNA), to serve as template for the synthesis of other polymers and macromolecules in biological processes having either a defined sequence of nucleotides (e.g., ribosomal RNA (rRNA), transfer RNA (tRNA) and mRNA) or a defined sequence of amino acids (e.g., polypeptide or protein) and the biological properties resulting therefrom.

“Expression” refers to the transcription and/or translation of a particular nucleotide sequence, such as a gene.

As used herein, the term “gene” broadly refers to an encoding nucleic acid sequence that can be transcribed into an RNA molecule, either a coding RNA molecule such as a mRNA which can be subsequently translated into a polypeptide or protein, or a non-coding RNA molecule such as a rRNA or a tRNA. “Transgene” refers in particular to a gene originating from one species which is to be introduced into an organism belonging to a different species. It should thus be noted that a gene may or may not encompass a coding sequence (or CDS), that is to say a nucleic acid sequence that actually codes for a protein. A gene, in particular a gene encompassing a CDS, may also preferably encompass untranslated transcribed regions (UTRs), such as a 3′-UTR and/or a 5′-UTR and other sequences, such as regulatory elements and/or introns, which are transcribed but not translated. Thus, as used herein, the term “gene” may refer to an encoding nucleic acid sequence comprising a coding sequence (or CDS) and at least one regulatory element that is transcribed but not translated, such as a 3′-UTR, a 5′-UTR and/or an intron.

As used herein, when referring to a given sequence (such as a miRNA target site), the expression “has/having a sequence as set forth in SEQ ID NO: X” means that the given sequence (such as a miRNA target site) comprises or consists of the sequence as set forth in SEQ ID NO: X.

“Identity” or “identical”, when used herein in a relationship between the sequences of two or more nucleic acids, refers to the degree of sequence relatedness between said nucleic acids, as determined by the number of matches between strings of two or more nucleotides. “Identity” measures the percent of identical matches between the smaller of two or more sequences with gap alignments (if any) addressed by a particular mathematical model or computer program (i.e., “algorithms”). Identity of related nucleic acid sequences can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988). Preferred methods for determining identity are designed to give the largest match between the sequences tested. Methods of determining identity are described in publicly available computer programs. Preferred computer program methods for determining identity between two sequences include the GCG program package, including GAP (Devereux et al., Nucleic Acids Res. 1984 Jan. 11; 12 (1 Pt 1): 387-95; Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., J. MoI. Biol. 215, 403-410 (1990)).

“Isolated” with reference to a nucleic acid refers to a nucleic acid altered or removed from the natural state. For example, a nucleic acid naturally present in a living organism is not “isolated” but the nucleic acid partially or completely separated from the coexisting materials of its natural state is “isolated”. An “isolated nucleic acid” is thus a nucleic acid that is substantially separated from other nucleic acid sequences, such as genomic DNA or RNA, as well as proteins or complexes such as ribosomes and polymerases, which naturally accompany a native sequence. An isolated nucleic acid can exist in substantially purified form, or can exist in a non-native environment such as, for example, a host cell. Typically, a preparation of an isolated nucleic acid may comprise the nucleic acid at least about 80% pure, at least about 85% pure, at least about 90% pure, at least about 95% pure, greater than about 95% pure, greater than about 96% pure, greater than about 97% pure, greater than about 98% pure, or greater than about 99% pure. Isolated nucleic acids thus include nucleic acids purified by standard purification methods, and encompass nucleic acid sequences that have been removed from their naturally occurring environment. Isolated nucleic acids also include chemically synthesized nucleic acids and nucleic acids biologically synthesized by heterologous systems.

“MicroRNAs” or “miRNAs” refer to endogenous small non-coding RNA molecules of about 18 to about 24 nucleotides that play a key role in the post-transcriptional regulation of gene expression in eukaryotic cells. A single miRNA may regulate up to hundreds of different mRNAs and most mRNAs are expected to be targeted by multiple miRNAs. miRNA genes are transcribed by RNA polymerases II or III and subsequently processed, giving rise to single-stranded mature miRNAs, which are incorporated into the RNA-induced silencing complex (RISC). As a central part of the RISC complex, the miRNA guides RISC to its mRNA targets, where the miRNA usually binds the 3′-untranslated region (3′UTR) of the mRNA transcript by partial complementary base pairing. Of note, complete base pairing must occur over a short length of 7 or 8 nucleotides, complementary to the so-called miRNA “seed” region located at positions 2-8 from the mature miRNA 5′-end. Gene silencing can occur through argonaute-2 (AG02)-mediated mRNA cleavage or through translational repression facilitated by AG01 to 4, with both ways finally leading to a reduction of the level of corresponding proteins. As used herein, “miRNA”, for example “miR183”, refers to a mature miRNA, for example to a mature miR183. By contrast, “precursor miRNA” or “pre-miRNA”, for example “precursor miR183”, refers to the hairpin precursor sequence from which is processed a mature miRNA.

“MicroRNA target site” or “miRNA target site” or “miR target site” as used herein refer to a nucleic acid sequence to which may bind a miRNA (i.e., a mature miRNA). As used herein, the terms “microRNA target site” or “miRNA target site” or “miR target site” encompass both the endogenous target sites that may be found in native transcripts and the artificial or engineered target sites (i.e., not naturally occurring target sites) that may be inserted as regulatory elements (or regulatory sequences) in vectors, in particular in expression vectors, for controlling the expression of a nucleic acid sequence of interest, such as a gene of interest. In particular, in a vector, miRNA target sites may be operably linked to or inserted in the sequence of a gene, in particular inserted in the transcribed sequence of a gene. By definition, a miRNA target site must comprise a nucleic acid sequence at least partially complementary to the corresponding miRNA, for example a nucleic acid sequence complementary to the corresponding miRNA over a length of at least 5 nucleotides, usually of 6-7 nucleotides. Thus, a miR183 target site must comprise a nucleic acid sequence at least partially complementary to miR183, for example a nucleic acid sequence complementary to miR183 over a length of at least 7-8 nucleotides. Similarly, a miR182 target site must comprise a nucleic acid sequence at least partially complementary to miR182, for example a nucleic acid sequence complementary to miR182 over a length of at least 7-8 nucleotides; and a miR96 target site must comprise a nucleic acid sequence at least partially complementary to miR96, for example a nucleic acid sequence complementary to miR96 over a length of at least 7-8 nucleotides. A miRNA target site, in particular an artificial or engineered miRNA target site (i.e., not naturally occurring target site) may also comprise or consist of a nucleic acid sequence complementary to the miRNA over the full length of the miRNA (i.e., over the 18 to 24 nucleotides of the miRNA). When inserted in a vector, a miR target site allows the binding of the corresponding miRNA and is thus capable of mediating miRNA induced silencing of the expression of a nucleic acid of interest, such as a gene of interest, upon introduction of the vector in a host cell expressing the miRNA. For example, when inserted in a vector, a miR183 target site allows the binding of miR183 and is thus capable of mediating miR183 induced silencing of the expression of a nucleic acid of interest, such as a gene of interest, upon introduction of the vector in a host cell expressing miR183. Similarly, when inserted in a vector, a miR182 target site allows the binding of miR182 and is thus capable of mediating miR182 induced silencing of the expression of a nucleic acid of interest, such as a gene of interest, upon introduction of the vector in a host cell expressing miR182; and when inserted in a vector, a miR96 target site allows the binding of miR96 and is thus capable of mediating miR96 induced silencing of the expression of a nucleic acid of interest, such as a gene of interest, upon introduction of the vector in a host cell expressing miR96. Methods for assessing whether a nucleic acid sequence may be a suitable miRNA target site (for example a miR183 target site, a miR182 target site, or a miR96 target site) are well known in the art. Examples of such methods are described in the experimental section hereinafter.

“MicroRNA target site of the miR183 family” or “miRNA target site of the miR183 family” or “miR target site of the miR183 family” as used herein refer to a nucleic acid sequence to which may bind a miRNA (i.e., a mature miRNA) belonging to the miR183 family (also sometimes referred to as miR183 cluster). The terms “microRNA target site of the miR183 family” or “miRNA target site of the miR183 family” or “miR target site of the miR183 family” thus refer to a nucleic acid sequence to which may bind miR183, miR182 and/or miR96.

“Nucleic acid” refers to a polymer of nucleotides (i.e., polynucleotides) covalently linked by phosphodiester bonds, such as deoxyribonucleic acids (DNA) or ribonucleic acids (RNA), in either single- or double-stranded form. A used herein, a nucleic acid may thus be single-stranded, partially double-stranded, or fully double-stranded. The nucleotides making up nucleic acids of the present disclosure may be unmodified (natural) nucleotides or non-natural or modified nucleotides. Unmodified (or natural or naturally occurring) nucleotides include adenosine monophosphate (AMP), deoxyadenosine monophosphate (dAMP), cytidine monophosphate (CMP), deoxycytidine monophosphate (dCMP), guanosine monophosphate (GMP), deoxyguanosine monophosphate (dGMP), thymidine monophosphate (TMP), deoxythymidine monophosphate (dTMP), and uridine monophosphate (UMP). The term “nucleic acid” also encompasses nucleic acids containing known analogues of natural nucleotides that have similar binding properties as the reference nucleic acid and are metabolized in a manner similar to naturally occurring nucleotides.

“Nucleic acid sequence” or “nucleotide sequence” refers to a contiguous sequence of nucleotides in a single nucleic acid. Unless otherwise indicated, a particular nucleic acid sequence also implicitly encompasses conservatively modified variants thereof (e.g., degenerate codon substitutions), alleles, orthologs, SNPs (single-nucleotide polymorphisms), and complementary sequences as well as the sequence explicitly indicated. Notably, a particular nucleic acid sequence described herein implicitly comprises its corresponding complementary sequence. It should be noted that a particular nucleic acid sequence described herein implicitly comprises the DNA sequence and the corresponding RNA sequence.

“Operatively linked” refers to functional linkage between a regulatory sequence and a heterologous nucleic acid sequence, e.g., a gene, resulting in a regulation of the expression of the latter by the former. For example, a first nucleic acid sequence is operably linked with a second nucleic acid sequence when the first nucleic acid sequence is placed in a functional relationship with the second nucleic acid sequence. In particular, a promoter is operably linked to a gene if the promoter affects the transcription or expression of the gene. Similarly, a regulatory sequence is operably linked to a gene if the regulatory sequence affects (i.e., either induces or inhibits (or represses)) the expression of the gene. Operably linked sequences can be contiguous with each other.

“Pharmaceutically acceptable excipient” or “pharmaceutically acceptable carrier” refers to an excipient or carrier that does not produce an adverse, allergic or other untoward reaction when administered to a mammal, preferably a human. It includes any and all solvents, such as, for example, dispersion media, coatings, antibacterial and antifungal agents, isotonic and absorption delaying agents. A pharmaceutically acceptable excipient or carrier refers to a non-toxic solid, semi-solid or liquid filler, diluent, encapsulating material or formulation auxiliary of any type. For human administration, preparations should meet sterility, pyrogenicity, general safety and purity standards as required by the regulatory offices such as the FDA (US Food and Drug Administration) or EMA (European Medicines Agency).

“Vector” refers to a vehicle by which a nucleic acid sequence (e.g., a DNA or RNA molecule), for example a nucleic acid encoding an RNA or a polypeptide or protein of interest, can be introduced into a host cell, so as to transform, transfect or transduce the host cell and promote expression (e.g., transcription and/or translation) of the introduced nucleic acid sequence.

“Expression vector” refers to a vector comprising regulatory elements (or regulatory sequences) operatively linked or to be operatively linked to a nucleic acid sequence of interest to be expressed, such as a gene of interest. An expression vector thus comprises sufficient cis-acting regulatory elements for controlling the expression of a nucleic acid sequence of interest (present or to be inserted in the expression vector); other elements that may be required for controlling the expression of the nucleic acid sequence of interest may be supplied by a host cell or an in vitro expression system (such as, for example, a miRNA that will bind to a miR target site). Cis-acting regulatory elements include for an example promoters and miR target sites such as the miR183 target site, miR182 target site and miR96 target site described herein.

### DETAILED DESCRIPTION

The human micro RNA 183 (miR183 or miR-183) belongs to the miR183 family (also sometimes referred to as miR183 cluster), which consists of 3 homologous miRNA: miR183 (or miR-183), miR96 (or miR-96) and miR182 (or miR-182). The miRNAs of the miR183 family are notably required for the proper development of the sensory organs. In particular, the miRNAs of the miR183 family are expressed in sensory neurons and hair cells in vertebrates and sensory cells of all species of animals. Among vertebrates, the miRNAs of the miR183 family are expressed in the olfactory epithelium, eye, neuromast and ear.

The human miR-183 gene consists of one exon on chromosome 7q32.2. The mature miR183 results from the processing of a hairpin precursor, called precursor miR183 or pre-miR183. The human precursor miR183 is 110 nucleotides long and has a sequence as set forth in SEQ ID NO: 2, which is referenced as NR_029615.1 in the NCBI databases.

Processing of the hairpin precursor miR183, which folds into a stem-loop structure, gives rise to a mature miR183. The human mature miR183 called miR183-5p (or hsa-miR183-5p or hsa-miR-183-5p) is 22 nucleotides long and has a sequence as set forth in SEQ ID NO: 3, which is referenced as MIMAT0000261 in the miRBase (https://www.mirbase.org). The sequence of miR183-5p corresponds to nucleotides 27 to 48 of the human precursor miR183 of SEQ ID NO: 2. The human mature miR183 called miR183-3p (or hsa-miR183-3p or hsa-miR-183-3p) is also 22 nucleotides long and has a sequence as set forth in SEQ ID NO: 4, which is referenced as MIMAT0004560 in the miRBase. The sequence of miR183-3p corresponds to nucleotides 66 to 87 of the human precursor miR183 of SEQ ID NO: 2. As used herein, the term “mature miR183” (or “mature miR-183)” encompasses both miR183-5p and miR183-3p.

In vivo, a mature miR183 can bind to target mRNAs comprising a short sequence complementary to the seed region of said mature miR183. For example, the seed region of hsa-miR183-5p is AUGGCAC, corresponding to nucleotides 2 to 8 of hsa-miR183-5p (SEQ ID NO: 3).

The human miR-182 gene consists of one exon on chromosome 7q32.2. The mature miR182 results from the processing of a hairpin precursor, called precursor miR182 or pre-miR182. The human precursor miR182 is 110 nucleotides long and has a sequence as set forth in SEQ ID NO: 22, which is referenced as NR_029614.1 in the NCBI databases.

Processing of the hairpin precursor miR182, which folds into a stem-loop structure, gives rise to a mature miR182. The human mature miR182 called miR182-5p (or hsa-miR182-5p or hsa-miR-182-5p) is 24 nucleotides long and has a sequence as set forth in SEQ ID NO: 11, which is referenced as MIMAT0000259 in the miRBase (https://www.mirbase.org). The sequence of miR182-5p corresponds to nucleotides 23 to 46 of the human precursor miR182 of SEQ ID NO: 22. The human mature miR182 called miR182-3p (or hsa-miR182-3p or hsa-miR-182-3p) is 21 nucleotides long and has a sequence as set forth in SEQ ID NO: 13, which is referenced as MIMAT0000260 in the miRBase. The sequence of miR182-3p corresponds to nucleotides 67 to 87 of the human precursor miR182 of SEQ ID NO: 22. As used herein, the term “mature miR182” (or “mature miR-182)” encompasses both miR182-5p and miR182-3p.

In vivo, a mature miR182 can bind to target mRNAs comprising a short sequence complementary to the seed region of said mature miR182. For example, the seed region of hsa-miR182-5p is UUGGCAA, corresponding to nucleotides 2 to 8 of hsa-miR182-5p (SEQ ID NO: 11).

The human miR-96 gene consists of one exon on chromosome 7q32.2. The mature miR96 results from the processing of a hairpin precursor, called precursor miR96 or pre-miR96. The human precursor miR96 is 78 nucleotides long and has a sequence as set forth in SEQ ID NO: 25, which is referenced as NR_029512.1 in the NCBI databases.

Processing of the hairpin precursor miR96, which folds into a stem-loop structure, gives rise to a mature miR96. The human mature miR96 called miR96-5p (or hsa-miR96-5p or hsa-miR-96-5p) is 23 nucleotides long and has a sequence as set forth in SEQ ID NO: 28, which is referenced as MIMAT0000095 in the miRBase (https://www.mirbase.org). The sequence of miR96-5p corresponds to nucleotides 9 to 31 of the human precursor miR96 of SEQ ID NO: 25. The human mature miR96 called miR96-3p (or hsa-miR96-3p or hsa-miR-96-3p) is 22 nucleotides long and has a sequence as set forth in SEQ ID NO: 30, which is referenced as MIMAT0004510 in the miRBase. The sequence of miR96-3p corresponds to nucleotides 52 to 73 of the human precursor miR96 of SEQ ID NO: 25. As used herein, the term “mature miR96” (or “mature miR-96)” encompasses both miR96-5p and miR96-3p.

In vivo, a mature miR96 can bind to target mRNAs comprising a short sequence complementary to the seed region of said mature miR96. For example, the seed region of hsa-miR96-5p is UUGGCAC, corresponding to nucleotides 2 to 8 of hsa-miR96-5p (SEQ ID NO: 28).

As detailed in the experimental section hereinafter, the Inventors have surprisingly demonstrated that, when inserted in a vector, a miR183 target site of 110 nucleotides long having a sequence as set forth in SEQ ID NO: 1, which is complementary to the human sequence of the precursor miR183 (i.e., SEQ ID NO: 2), can successfully be used as a regulatory element to control the expression of a nucleic acid of interest in cells expressing miR183. As indicated above, the miR183 target site having a sequence that is complementary to the human sequence of the precursor miR183 (i.e., SEQ ID NO: 2) is sometimes referred herein as the “precursor miR183 target site”. Upon introduction in cells, the mirR183 target site comprised within the expression vector is transcribed along with the nucleic acid of interest and is thus present in the resulting mRNA. The mir183 target site of 110 nucleotides long having a sequence as set forth in SEQ ID NO: 1 is expected to fold into a stem-loop structure. The Inventors have surprisingly shown that, against expectation, the stem-loop structure does not prevent the binding of miR183 to the mir183 target site of SEQ ID NO: 1.

Similarly, the Inventors have demonstrated that, when inserted in a vector, a miR182 target site of 110 nucleotides long having a sequence as set forth in SEQ ID NO: 21, which is complementary to the human sequence of the precursor miR182 (i.e., SEQ ID NO: 22), can successfully be used as a regulatory element to control the expression of a nucleic acid of interest in cells expressing miR182. As indicated above, the miR182 target site having a sequence that is complementary to the human sequence of the precursor miR182 (i.e., SEQ ID NO: 22) is sometimes referred herein as the “precursor miR182 target site”. Upon introduction in cells, the mirR182 target site comprised within the expression vector is transcribed along with the nucleic acid of interest and is thus present in the resulting mRNA. The Inventors have also demonstrated that, when inserted in a vector, a miR96 target site of 78 nucleotides long having a sequence as set forth in SEQ ID NO: 24, which is complementary to the human sequence of the precursor miR96 (i.e., SEQ ID NO: 25), can successfully be used as a regulatory element to control the expression of a nucleic acid of interest in cells expressing miR96. As indicated above, the miR96 target site having a sequence that is complementary to the human sequence of the precursor miR96 (i.e., SEQ ID NO: 25) is sometimes referred herein as the “precursor miR96 target site”. Upon introduction in cells, the mirR96 target site comprised within the expression vector is transcribed along with the nucleic acid of interest and is thus present in the resulting mRNA. The mir182 target site of 110 nucleotides long having a sequence as set forth in SEQ ID NO: 21 and the mir96 target site of 78 nucleotides long having a sequence as set forth in SEQ ID NO: 24 are also expected to fold into a stem-loop structure. The Inventors have surprisingly shown that, against expectation, the stem-loop structure prevents neither the binding of miR182 to the mir182 target site of SEQ ID NO: 21 nor the binding of miR96 to the mir96 target site of SEQ ID NO: 24.

The Inventors have also shown that inserting several copies of the miR183 target site of SEQ ID NO: 1 leads to a stronger repression of the expression of the nucleic acid. Unexpectedly, the repression of the nucleic acid expression induced through the use of the miR183 target site of SEQ ID NO: 1 is significantly stronger than the repression induced through the use of a shorter target site, such as a miR183 target site having a sequence as set forth in SEQ ID NO: 5, which is complementary to the sequence of hsa-miR183-5p (i.e., SEQ ID NO: 3).

As illustrated in the experimental section hereinafter, methods for assessing whether a nucleic acid sequence may be a suitable miR target site, for example a suitable miR183 target site, include inserting the assessed nucleic acid sequence in a vector comprising a reporter gene, such as the gene encoding for the GFP protein (green fluorescent protein), under the control of a promoter such as a ubiquitous or constitutive promoter. The assessed nucleic acid sequence may be operably linked to the gene or inserted in the gene, for example in the 3′-UTR of the gene. The vector (for example a plasmid) comprising the assessed nucleic acid sequence and the reporter gene is then introduced in a host cell expressing miR183, such as for example HEK293 cells. Inhibition of the expression of the reporter gene in the host cell, in particular in comparison with a control condition wherein the vector comprises only the reporter gene under the control of the promoter, indicates that the assessed nucleic acid sequence is a suitable miR183 target site. A similar method may be used for assessing whether a nucleic acid sequence may be a suitable miR182 target site or a suitable miR96 target site.

A first aspect of the present invention is thus an isolated nucleic acid sequence comprising or consisting of at least two copies of a miR target site of the miR183 family, said miR target site having a sequence as set forth in SEQ ID NO: 1, SEQ ID NO: 21, or SEQ ID NO: 24, or a sequence having at least 70%, 75%, 80%, 85%, 90%, 95%, 96%, 97%, 98%, or 99%, or more identity with any one of SEQ ID NO: 1, SEQ ID NO: 21, or SEQ ID NO: 24.

The isolated nucleic acid sequence as described herein may comprise or consist of at least two copies of a miR183 target site having a sequence as set forth in SEQ ID NO: 1, or a sequence having at least 70%, 75%, 80%, 85%, 90%, 95%, 96%, 97%, 98%, or 99%, or more identity with SEQ ID NO: 1. In some embodiments, the mirR183 target site having a sequence with at least 70%, 75%, 80%, 85%, 90%, 95%, 96%, 97%, 98%, or 99%, or more identity with SEQ ID NO: 1 as described herein is a functional mirR183 target site, that is to say it allows the binding of miR183.

In some embodiments, the mirR183 target site having a sequence with at least 70%, 75%, 80%, 85%, 90%, 95%, 96%, 97%, 98%, or 99%, or more identity with SEQ ID NO: 1 has a length of at least 100 nucleotides. In some embodiments, the mirR183 target site having a sequence with at least 70%, 75%, 80%, 85%, 90%, 95%, 96%, 97%, 98%, or 99%, or more identity with SEQ ID NO: 1 has a length of at least 60, 65, 70, 75, 80, 85, 90, 95, 100, or 105 nucleotides. In some embodiments, the mirR183 target site having a sequence with at least 70%, 75%, 80%, 85%, 90%, 95%, 96%, 97%, 98%, or 99%, or more identity with SEQ ID NO: 1 has a length of 90, 91, 92, 93, 94, 95, 96, 97, 98, 99, 100, 101, 102, 103, 104, 105, 106, 107, 108, 109, or 110 nucleotides.

In some embodiments, the mirR183 target site has a sequence as set forth in SEQ ID NO: 1 with 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60, or more nucleotides being substituted by a different nucleotide with reference to the corresponding nucleotide(s) of SEQ ID NO: 1. In some embodiments, the mirR183 target site has a sequence as set forth in SEQ ID NO: 1 with at most 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 nucleotide(s) being substituted by a different nucleotide with reference to the corresponding nucleotide(s) of SEQ ID NO: 1. In some embodiments, such a mirR183 target site having a sequence as set forth in SEQ ID NO: 1 with nucleotide substitution(s) is a functional mirR183 target site, that is to say it allows the binding of miR183.

The isolated nucleic acid sequence as described herein may comprise or consist of at least two copies of a miR182 target site having a sequence as set forth in SEQ ID NO: 21, or a sequence having at least 70%, 75%, 80%, 85%, 90%, 95%, 96%, 97%, 98%, or 99%, or more identity with SEQ ID NO: 21. In some embodiments, the mirR182 target site having a sequence with at least 70%, 75%, 80%, 85%, 90%, 95%, 96%, 97%, 98%, or 99%, or more identity with SEQ ID NO: 21 as described herein is a functional mirR182 target site, that is to say it allows the binding of miR182.

In some embodiments, the mirR182 target site having a sequence with at least 70%, 75%, 80%, 85%, 90%, 95%, 96%, 97%, 98%, or 99%, or more identity with SEQ ID NO: 21 has a length of at least 100 nucleotides. In some embodiments, the mirR182 target site having a sequence with at least 70%, 75%, 80%, 85%, 90%, 95%, 96%, 97%, 98%, or 99%, or more identity with SEQ ID NO: 21 has a length of at least 60, 65, 70, 75, 80, 85, 90, 95, 100, or 105 nucleotides. In some embodiments, the mirR182 target site having a sequence with at least 70%, 75%, 80%, 85%, 90%, 95%, 96%, 97%, 98%, or 99%, or more identity with SEQ ID NO: 21 has a length of 90, 91, 92, 93, 94, 95, 96, 97, 98, 99, 100, 101, 102, 103, 104, 105, 106, 107, 108, 109, or 110 nucleotides.

In some embodiments, the mirR182 target site has a sequence as set forth in SEQ ID NO: 21 with 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60, or more nucleotides being substituted by a different nucleotide with reference to the corresponding nucleotide(s) of SEQ ID NO: 21. In some embodiments, the mirR182 target site has a sequence as set forth in SEQ ID NO: 21 with at most 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 nucleotide(s) being substituted by a different nucleotide with reference to the corresponding nucleotide(s) of SEQ ID NO: 21. In some embodiments, such a mirR182 target site having a sequence as set forth in SEQ ID NO: 21 with nucleotide substitution(s) is a functional mirR182 target site, that is to say it allows the binding of miR182.

The isolated nucleic acid sequence as described herein may comprise or consist of at least two copies of a mi96 target site having a sequence as set forth in SEQ ID NO: 24, or a sequence having at least 70%, 75%, 80%, 85%, 90%, 95%, 96%, 97%, 98%, or 99%, or more identity with SEQ ID NO: 24. In some embodiments, the mirR96 target site having a sequence with at least 70%, 75%, 80%, 85%, 90%, 95%, 96%, 97%, 98%, or 99%, or more identity with SEQ ID NO: 24 as described herein is a functional mirR183 target site, that is to say it allows the binding of miR96.

In some embodiments, the mirR96 target site having a sequence with at least 70%, 75%, 80%, 85%, 90%, 95%, 96%, 97%, 98%, or 99%, or more identity with SEQ ID NO: 24 has a length of at least 70 nucleotides. In some embodiments, the mir96 target site having a sequence with at least 70%, 75%, 80%, 85%, 90%, 95%, 96%, 97%, 98%, or 99%, or more identity with SEQ ID NO: 24 has a length of at least 40, 45, 50, 55, 60, or 65 nucleotides. In some embodiments, the mir96 target site having a sequence with at least 70%, 75%, 80%, 85%, 90%, 95%, 96%, 97%, 98%, or 99%, or more identity with SEQ ID NO: 24 has a length of 58, 59, 60, 61, 62, 63, 64, 65, 66, 67, 68, 69, 70, 71, 72, 73, 74, 75, 76, 77, or 78 nucleotides.

In some embodiments, the mirR96 target site has a sequence as set forth in SEQ ID NO: 24 with 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35, or more nucleotides being substituted by a different nucleotide with reference to the corresponding nucleotide(s) of SEQ ID NO: 24. In some embodiments, the mirR96 target site has a sequence as set forth in SEQ ID NO: 24 with at most 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 nucleotide(s) being substituted by a different nucleotide with reference to the corresponding nucleotide(s) of SEQ ID NO: 24. In some embodiments, such a mirR96 target site having a sequence as set forth in SEQ ID NO: 24 with nucleotide substitution(s) is a functional mirR96 target site, that is to say it allows the binding of miR96.

As used herein, the expression “at least two copies” include two, three, four, five, six, seven, eight, nine, ten, or more copies of the miR target site of the miR183 family as described herein (such as, for example, of the miR183 target site having a sequence as set forth in SEQ ID NO: 1, or a sequence having at least 70%, 75%, 80%, 85%, 90%, 95%, 96%, 97%, 98%, or 99%, or more identity with SEQ ID NO: 1, as described herein).

In some embodiments, the isolated nucleic acid sequence comprises or consists of two to six copies of the miR target site of the miR183 family as described herein (such as, for example, of the miR183 target site having a sequence as set forth in SEQ ID NO: 1, or a sequence having at least 70%, 75%, 80%, 85%, 90%, 95%, 96%, 97%, 98%, or 99%, or more identity with SEQ ID NO: 1, as described herein). The isolated nucleic acid sequence may thus comprise or consist of two, three, four, five, or six copies of the miR target site of the miR183 family as described herein (such as, for example, of the miR183 target site having a sequence as set forth in SEQ ID NO: 1, or a sequence having at least 70%, 75%, 80%, 85%, 90%, 95%, 96%, 97%, 98%, or 99%, or more identity with SEQ ID NO: 1, as described herein).

In some embodiments, the isolated nucleic acid sequence comprises or consists of three copies of the miR target site of the miR183 family as described herein. The isolated nucleic acid sequence may thus comprise or consist of a sequence as set forth in SEQ ID NO: 7, SEQ ID NO: 23, or SEQ ID NO: 26, or a sequence having at least 70%, 75%, 80%, 85%, 90%, 95%, 96%, 97%, 98%, or 99%, or more identity with any one of SEQ ID NO: 7, SEQ ID NO: 23, or SEQ ID NO: 26.

In particular, the isolated nucleic acid sequence may comprise or consist of three copies of the miR183 target site having a sequence as set forth in SEQ ID NO: 1, or a sequence having at least 70%, 75%, 80%, 85%, 90%, 95%, 96%, 97%, 98%, or 99%, or more identity with SEQ ID NO: 1, as described herein. The isolated nucleic acid sequence may thus comprise or consist of a sequence as set forth in SEQ ID NO: 7, or a sequence having at least 70%, 75%, 80%, 85%, 90%, 95%, 96%, 97%, 98%, or 99%, or more identity with SEQ ID NO: 7. In some embodiments, the isolated nucleic acid sequence having a sequence with at least 70%, 75%, 80%, 85%, 90%, 95%, 96%, 97%, 98%, or 99%, or more identity with SEQ ID NO: 7 as described herein corresponds to a functional mirR183 target site, that is to say it allows the binding of miR183.

In some embodiments, the isolated nucleic acid sequence having a sequence with at least 70%, 75%, 80%, 85%, 90%, 95%, 96%, 97%, 98%, or 99%, or more identity with SEQ ID NO: 7 has a length of at least 300 nucleotides. In some embodiments, the isolated nucleic acid sequence having a sequence with at least 70%, 75%, 80%, 85%, 90%, 95%, 96%, 97%, 98%, or 99%, or more identity with SEQ ID NO: 7 has a length of at least 180, 190, 200, 210, 220, 230, 240, 250, 260, 270, 280, 290, 300, 305, 310, 315, 320, 325, or 330 nucleotides. In some embodiments, the isolated nucleic acid sequence having a sequence with at least 70%, 75%, 80%, 85%, 90%, 95%, 96%, 97%, 98%, or 99%, or more identity with SEQ ID NO: 7 has a length of 300, 305, 310, 315, 320, 325, or 330 nucleotides.

In some embodiments the isolated nucleic acid sequence has a sequence as set forth in SEQ ID NO: 7 with 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, or more nucleotides being substituted by a different nucleotide with reference to the corresponding nucleotide(s) of SEQ ID NO: 7. In some embodiments the isolated nucleic acid sequence has a sequence as set forth in SEQ ID NO: 7 with at most 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 nucleotide(s) being substituted by a different nucleotide with reference to the corresponding nucleotide(s) of SEQ ID NO: 7. In some embodiments, such an isolated nucleic acid sequence having a sequence as set forth in SEQ ID NO: 7 with nucleotide substitution(s) corresponds to a functional mirR183 target site, that is to say it allows the binding of miR183.

The isolated nucleic acid sequence may comprise or consist of three copies of the miR182 target site having a sequence as set forth in SEQ ID NO: 21, or a sequence having at least 70%, 75%, 80%, 85%, 90%, 95%, 96%, 97%, 98%, or 99%, or more identity with SEQ ID NO: 21, as described herein. The isolated nucleic acid sequence may thus comprise or consist of a sequence as set forth in SEQ ID NO: 23, or a sequence having at least 70%, 75%, 80%, 85%, 90%, 95%, 96%, 97%, 98%, or 99%, or more identity with SEQ ID NO: 23. In some embodiments, the isolated nucleic acid sequence having a sequence with at least 70%, 75%, 80%, 85%, 90%, 95%, 96%, 97%, 98%, or 99%, or more identity with SEQ ID NO: 23 as described herein corresponds to a functional mirR182 target site, that is to say it allows the binding of miR182.

In some embodiments, the isolated nucleic acid sequence having a sequence with at least 70%, 75%, 80%, 85%, 90%, 95%, 96%, 97%, 98%, or 99%, or more identity with SEQ ID NO: 23 has a length of at least 300 nucleotides. In some embodiments, the isolated nucleic acid sequence having a sequence with at least 70%, 75%, 80%, 85%, 90%, 95%, 96%, 97%, 98%, or 99%, or more identity with SEQ ID NO: 23 has a length of at least 180, 190, 200, 210, 220, 230, 240, 250, 260, 270, 280, 290, 300, 305, 310, 315, 320, 325, or 330 nucleotides. In some embodiments, the isolated nucleic acid sequence having a sequence with at least 70%, 75%, 80%, 85%, 90%, 95%, 96%, 97%, 98%, or 99%, or more identity with SEQ ID NO: 23 has a length of 300, 305, 310, 315, 320, 325, or 330 nucleotides.

In some embodiments the isolated nucleic acid sequence has a sequence as set forth in SEQ ID NO: 23 with 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, or more nucleotides being substituted by a different nucleotide with reference to the corresponding nucleotide(s) of SEQ ID NO: 23. In some embodiments the isolated nucleic acid sequence has a sequence as set forth in SEQ ID NO: 23 with at most 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 nucleotide(s) being substituted by a different nucleotide with reference to the corresponding nucleotide(s) of SEQ ID NO: 23. In some embodiments, such an isolated nucleic acid sequence having a sequence as set forth in SEQ ID NO: 23 with nucleotide substitution(s) corresponds to a functional mirR182 target site, that is to say it allows the binding of miR182.

The isolated nucleic acid sequence may comprise or consist of three copies of the miR96 target site having a sequence as set forth in SEQ ID NO: 24, or a sequence having at least 70%, 75%, 80%, 85%, 90%, 95%, 96%, 97%, 98%, or 99%, or more identity with SEQ ID NO: 24, as described herein. The isolated nucleic acid sequence may thus comprise or consist of a sequence as set forth in SEQ ID NO: 26, or a sequence having at least 70%, 75%, 80%, 85%, 90%, 95%, 96%, 97%, 98%, or 99%, or more identity with SEQ ID NO: 26. In some embodiments, the isolated nucleic acid sequence having a sequence with at least 70%, 75%, 80%, 85%, 90%, 95%, 96%, 97%, 98%, or 99%, or more identity with SEQ ID NO: 26 as described herein corresponds to a functional mirR96 target site, that is to say it allows the binding of miR96.

In some embodiments, the isolated nucleic acid sequence having a sequence with at least 70%, 75%, 80%, 85%, 90%, 95%, 96%, 97%, 98%, or 99%, or more identity with SEQ ID NO: 26 has a length of at least 210 nucleotides. In some embodiments, the isolated nucleic acid sequence having a sequence with at least 70%, 75%, 80%, 85%, 90%, 95%, 96%, 97%, 98%, or 99%, or more identity with SEQ ID NO: 26 has a length of at least 160, 170, 180, 190, 200, 205, 210, 215, 220, 225, 230, or 234 nucleotides. In some embodiments, the isolated nucleic acid sequence having a sequence with at least 70%, 75%, 80%, 85%, 90%, 95%, 96%, 97%, 98%, or 99%, or more identity with SEQ ID NO: 26 has a length of 200, 205, 210, 215, 220, 225, 230, or 234 nucleotides.

In some embodiments the isolated nucleic acid sequence has a sequence as set forth in SEQ ID NO: 26 with 1, 2, 3, 4, 5, 6, 7, 8, 9, 10, 11, 12, 13, 14, 15, 16, 17, 18, 19, or 20, or more nucleotides being substituted by a different nucleotide with reference to the corresponding nucleotide(s) of SEQ ID NO: 26. In some embodiments the isolated nucleic acid sequence has a sequence as set forth in SEQ ID NO: 26 with at most 20, 19, 18, 17, 16, 15, 14, 13, 12, 11, 10, 9, 8, 7, 6, 5, 4, 3, 2, or 1 nucleotide(s) being substituted by a different nucleotide with reference to the corresponding nucleotide(s) of SEQ ID NO: 26. In some embodiments, such an isolated nucleic acid sequence having a sequence as set forth in SEQ ID NO: 26 with nucleotide substitution(s) corresponds to a functional mirR96 target site, that is to say it allows the binding of miR96.

In some embodiments, in the isolated nucleic acid sequence as described herein, the copies of the miR target site of the miR183 family as described herein (such as, for example; of the miR183 target site) are separated by a spacer. As used herein, “spacer” refers to a non-coding sequence. In some embodiments, the spacer is characterized by a length ranging from about 5 nucleotides to about 25 nucleotides, preferably from about 10 nucleotides to about 20 nucleotides, more preferably of about 20 nucleotides. Spacers are commonly used in the field and are well-known to the skilled artisan. For example, spacers are described in Hammarsten et al. Herpes simplex virus: selection of origins of DNA replication. Nucleic Acids Res. 1997 May 1; 25 (9): 1753-60). Examples of spacers include spacers having a sequence as set forth in SEQ ID NO: 14 (ATAACTAAAAGATTCGGA), in SEQ ID NO: 15 (AATATATATATATTATTA), in SEQ ID NO: 16 (AAAAACATATAAAATAAT), or in SEQ ID NO: 17 (CTTTCTTTTCCCAATTTT). In some embodiments, the spacer has a sequence as set forth in SEQ ID NO: 14.

In some embodiments, the isolated nucleic acid sequence is a single-stranded sequence. In some embodiments, the isolated nucleic acid sequence is a double-stranded sequence.

In some embodiments, the isolated nucleic acid sequence as described herein further comprises at least one copy of another miR target site. In some embodiments, the isolated nucleic acid sequence comprises or consists of:
at least two copies of a miR183 target site having a sequence as set forth in SEQ ID NO: 1, or a sequence having at least 70%, 75%, 80%, 85%, 90%, 95%, 96%, 97%, 98%, or 99%, or more identity with SEQ ID NO: 1, as described herein; or at least two copies of a miR182 target site having a sequence as set forth in SEQ ID NO: 21, or a sequence having at least 70%, 75%, 80%, 85%, 90%, 95%, 96%, 97%, 98%, or 99%, or more identity with SEQ ID NO: 21, as described herein; or at least two copies of a miR96 target site having a sequence as set forth in SEQ ID NO: 24, or a sequence having at least 70%, 75%, 80%, 85%, 90%, 95%, 96%, 97%, 98%, or 99%, or more identity with SEQ ID NO: 24, as described herein; and
at least one copy of another miR target site.

For example, the other miR target site may be a miR target site recognized by a miRNA expressed in sensory neurons and/or hair cells, such as hair cells of the inner ear. The other miR target may be a miR182 target site, a miR96 target site, a miR194 target site, a miR140 target site, a miR18a target site, a miR99a target site, a miR30b target site, a miR15a target site, or a miR210 target site. In some embodiments, the other miR target site is a miR182 target site.

The miR182 target site may be a miR182 target site having a sequence complementary to a mature miR182. For example, the miR182 target site may have a sequence as set forth in SEQ ID NO: 10, or a sequence having at least 70%, 75%, 80%, 85%, 90%, 95%, 96%, 97%, 98%, or 99%, or more identity with SEQ ID NO: 10. The sequence of SEQ ID NO: 10 is complementary to the sequence of hsa-miR182-5p. Hsa-miR182-5p is 24 nucleotides long and has a sequence as set forth in SEQ ID NO: 11, which is referenced as MIMAT0000259 in the miRBase as indicated above. The mature miR182 target site may have a sequence as set forth in SEQ ID NO: 12, or a sequence having at least 70%, 75%, 80%, 85%, 90%, 95%, 96%, 97%, 98%, or 99%, or more identity with SEQ ID NO: 12. The sequence of SEQ ID NO: 12 is complementary to the sequence of hsa-miR182-3p. Hsa-miR182-3p is 21 nucleotides long and has a sequence as set forth in SEQ ID NO: 13, which is referenced as MIMAT0000260 in the miRBase as indicated above.

In some embodiments, the isolated nucleic acid sequence as described herein further comprises at least one copy of another miR target site of the miR183 family as described herein. For example, the isolated nucleic acid sequence may comprise or consist of:
at least two copies of a miR183 target site having a sequence as set forth in SEQ ID NO: 1, or a sequence having at least 70%, 75%, 80%, 85%, 90%, 95%, 96%, 97%, 98%, or 99%, or more identity with SEQ ID NO: 1, as described herein; and
at least one copy of a miR182 target site having a sequence as set forth in SEQ ID NO: 21, or a sequence having at least 70%, 75%, 80%, 85%, 90%, 95%, 96%, 97%, 98%, or 99%, or more identity with SEQ ID NO: 21, and/or at least one copy of a miR96 target site having a sequence as set forth in SEQ ID NO: 24, or a sequence having at least 70%, 75%, 80%, 85%, 90%, 95%, 96%, 97%, 98%, or 99%, or more identity with SEQ ID NO: 24.

The isolated nucleic acid sequence may comprise or consist of:
at least two copies of a miR182 target site having a sequence as set forth in SEQ ID NO: 21, or a sequence having at least 70%, 75%, 80%, 85%, 90%, 95%, 96%, 97%, 98%, or 99%, or more identity with SEQ ID NO: 21, as described herein; and
at least one copy of a miR183 target site having a sequence as set forth in SEQ ID NO: 1, or a sequence having at least 70%, 75%, 80%, 85%, 90%, 95%, 96%, 97%, 98%, or 99%, or more identity with SEQ ID NO: 1, and/or at least one copy of a miR96 target site having a sequence as set forth in SEQ ID NO: 24, or a sequence having at least 70%, 75%, 80%, 85%, 90%, 95%, 96%, 97%, 98%, or 99%, or more identity with SEQ ID NO: 24.

The isolated nucleic acid sequence may comprise or consist of:
at least two copies of a miR96 target site having a sequence as set forth in SEQ ID NO: 24, or a sequence having at least 70%, 75%, 80%, 85%, 90%, 95%, 96%, 97%, 98%, or 99%, or more identity with SEQ ID NO: 24, as described herein; and
at least one copy of a miR183 target site having a sequence as set forth in SEQ ID NO: 1, or a sequence having at least 70%, 75%, 80%, 85%, 90%, 95%, 96%, 97%, 98%, or 99%, or more identity with SEQ ID NO: 1, and/or at least one copy of a miR182 target site having a sequence as set forth in SEQ ID NO: 21, or a sequence having at least 70%, 75%, 80%, 85%, 90%, 95%, 96%, 97%, 98%, or 99%, or more identity with SEQ ID NO: 21.

Another aspect of the present invention is an isolated nucleic acid coding sequence (also referred to as isolated coding sequence) comprising at least one copy of a miR target site of the miR183 family, said miR target site having a sequence as set forth in SEQ ID NO: 1, SEQ ID NO: 21, or SEQ ID NO: 24, or a sequence having at least 70%, 75%, 80%, 85%, 90%, 95%, 96%, 97%, 98%, or 99%, or more identity with any one of SEQ ID NO: 1, SEQ ID NO: 21, or SEQ ID NO: 24.

The isolated coding sequence may comprise at least one copy of a miR183 target site having a sequence as set forth in SEQ ID NO: 1, or a sequence having at least 70%, 75%, 80%, 85%, 90%, 95%, 96%, 97%, 98%, or 99%, or more identity with SEQ ID NO: 1, as described herein. The isolated coding sequence may comprise at least one copy of a miR182 target site having a sequence as set forth in SEQ ID NO: 21, or a sequence having at least 70%, 75%, 80%, 85%, 90%, 95%, 96%, 97%, 98%, or 99%, or more identity with SEQ ID NO: 21, as described herein. The isolated coding sequence may comprise at least one copy of a miR96 target site having a sequence as set forth in SEQ ID NO: 24, or a sequence having at least 70%, 75%, 80%, 85%, 90%, 95%, 96%, 97%, 98%, or 99%, or more identity with SEQ ID NO: 24, as described herein.

In some embodiments, the isolated nucleic acid coding sequence encodes for a polynucleotide or a protein. In some embodiments, the isolated coding sequence is a cDNA sequence. In some embodiments, the isolated coding sequence is an RNA coding sequence. For example, in some embodiments, the isolated coding sequence is a mRNA sequence. In some embodiments, the at least one copy of a miR target site of the miR183 family as described herein is inserted in an untranslated region of the isolated coding sequence as described herein, such as, for example, in the 5′-UTR or in the 3′-UTR of the isolated coding sequence.

In some embodiments, the isolated nucleic acid coding sequence comprises at least two copies of a miR target site of the miR183 family as described herein (such as, for example, of the miR183 target site having a sequence as set forth in SEQ ID NO: 1, or a sequence having at least 70%, 75%, 80%, 85%, 90%, 95%, 96%, 97%, 98%, or 99%, or more identity with SEQ ID NO: 1, as described herein).

In some embodiments, the isolated nucleic acid coding sequence comprises two to six copies of the miR target site of the miR183 family as described herein (such as, for example, of the miR183 target site having a sequence as set forth in SEQ ID NO: 1, or a sequence having at least 70%, 75%, 80%, 85%, 90%, 95%, 96%, 97%, 98%, or 99%, or more identity with SEQ ID NO: 1, as described herein).

In some embodiments, the isolated nucleic acid coding sequence comprises three copies of the miR target site of the miR183 family as described herein. The isolated nucleic acid sequence may thus comprise or consist of a sequence as set forth in SEQ ID NO: 7, SEQ ID NO: 23, or SEQ ID NO: 26, or a sequence having at least 70%, 75%, 80%, 85%, 90%, 95%, 96%, 97%, 98%, or 99%, or more identity with any one of SEQ ID NO: 7, SEQ ID NO: 23, or SEQ ID NO: 26.

In particular, the isolated nucleic acid coding sequence may comprise three copies of the miR183 target site having a sequence as set forth in SEQ ID NO: 1, or a sequence having at least 70%, 75%, 80%, 85%, 90%, 95%, 96%, 97%, 98%, or 99%, or more identity with SEQ ID NO: 1, as described herein. The isolated nucleic acid coding sequence may thus comprise a sequence as set forth in SEQ ID NO: 7, or a sequence having at least 70%, 75%, 80%, 85%, 90%, 95%, 96%, 97%, 98%, or 99%, or more identity with SEQ ID NO: 7, as described herein.

The isolated nucleic acid coding sequence may comprise three copies of the miR182 target site having a sequence as set forth in SEQ ID NO: 21, or a sequence having at least 70%, 75%, 80%, 85%, 90%, 95%, 96%, 97%, 98%, or 99%, or more identity with SEQ ID NO: 21, as described herein. The isolated nucleic acid coding sequence may thus comprise a sequence as set forth in SEQ ID NO: 23, or a sequence having at least 70%, 75%, 80%, 85%, 90%, 95%, 96%, 97%, 98%, or 99%, or more identity with SEQ ID NO: 23, as described herein.

The isolated nucleic acid coding sequence may comprise three copies of the miR96 target site having a sequence as set forth in SEQ ID NO: 24, or a sequence having at least 70%, 75%, 80%, 85%, 90%, 95%, 96%, 97%, 98%, or 99%, or more identity with SEQ ID NO: 24, as described herein. The isolated nucleic acid coding sequence may thus comprise a sequence as set forth in SEQ ID NO: 26, or a sequence having at least 70%, 75%, 80%, 85%, 90%, 95%, 96%, 97%, 98%, or 99%, or more identity with SEQ ID NO: 26, as described herein.

In some embodiments, the copies of the miR target site of the miR183 family as described herein (such as, for example, of the miR183 target site) are separated by a spacer as described herein. In some embodiments, the spacer has a sequence as set forth in SEQ ID NO: 14.

In some embodiments, the isolated nucleic acid coding sequence further comprises at least one copy of another miR target site. In some embodiments, the isolated nucleic acid coding sequence comprises:
at least one copy of a miR183 target site having a sequence as set forth in SEQ ID NO: 1, or a sequence having at least 70%, 75%, 80%, 85%, 90%, 95%, 96%, 97%, 98%, or 99%, or more identity with SEQ ID NO: 1, as described herein; or at least one copy of a miR182 target site having a sequence as set forth in SEQ ID NO: 21, or a sequence having at least 70%, 75%, 80%, 85%, 90%, 95%, 96%, 97%, 98%, or 99%, or more identity with SEQ ID NO: 21, as described herein; or at least one copy of a miR96 target site having a sequence as set forth in SEQ ID NO: 24, or a sequence having at least 70%, 75%, 80%, 85%, 90%, 95%, 96%, 97%, 98%, or 99%, or more identity with SEQ ID NO: 24, as described herein; and
at least one copy of another miR target site.

For example, the other miR target site may be a miR target site recognized by a miRNA expressed in sensory neurons and/or hair cells, such as hair cells of the inner ear. The other miR target may be a miR182 target site, a miR96 target site, a miR194 target site, a miR140 target site, a miR18a target site, a miR99a target site, a miR30b target site, a miR15a target site, or a miR210 target site. In some embodiments, the other miR target site is a miR182 target site.

The miR182 target site may have a sequence complementary to a mature miR182, for example a sequence as set forth in SEQ ID NO: 10, in SEQ ID NO: 12, or a sequence having at least 70%, 75%, 80%, 85%, 90%, 95%, 96%, 97%, 98%, or 99%, or more identity with SEQ ID NO: 10 or SEQ ID NO: 12.

In some embodiments, the isolated nucleic acid coding sequence further comprises at least one copy of another miR target site of the miR183 family as described herein. For example, the isolated nucleic acid coding sequence may comprise:
at least one copy of a miR183 target site having a sequence as set forth in SEQ ID NO: 1, or a sequence having at least 70%, 75%, 80%, 85%, 90%, 95%, 96%, 97%, 98%, or 99%, or more identity with SEQ ID NO: 1, as described herein; and
at least one copy of a miR182 target site having a sequence as set forth in SEQ ID NO: 21, or a sequence having at least 70%, 75%, 80%, 85%, 90%, 95%, 96%, 97%, 98%, or 99%, or more identity with SEQ ID NO: 21, as described herein, and/or at least one copy of a miR96 target site having a sequence as set forth in SEQ ID NO: 24, or a sequence having at least 70%, 75%, 80%, 85%, 90%, 95%, 96%, 97%, 98%, or 99%, or more identity with SEQ ID NO: 24, as described herein.

Alternatively, the isolated nucleic acid coding sequence may comprise at least one copy of a miR182 target site having a sequence as set forth in SEQ ID NO: 21, or a sequence having at least 70%, 75%, 80%, 85%, 90%, 95%, 96%, 97%, 98%, or 99%, or more identity with SEQ ID NO: 21, as described herein, and at least one copy of a miR96 target site having a sequence as set forth in SEQ ID NO: 24, or a sequence having at least 70%, 75%, 80%, 85%, 90%, 95%, 96%, 97%, 98%, or 99%, or more identity with SEQ ID NO: 24, as described herein.

Another aspect of the present invention is an expression cassette comprising a promoter, a nucleic acid sequence of interest, and a regulatory element (or regulatory sequence) comprising or consisting of at least one copy of a miR target site of the miR183 family, said miR target site having a sequence as set forth in SEQ ID NO: 1, SEQ ID NO: 21, or SEQ ID NO: 24, or a sequence having at least 70%, 75%, 80%, 85%, 90%, 95%, 96%, 97%, 98%, or 99%, or more identity with any one of SEQ ID NO: 1, SEQ ID NO: 21, or SEQ ID NO: 24.

The regulatory element (or regulatory sequence) may comprise or consist of at least one copy of a miR183 target site having a sequence as set forth in SEQ ID NO: 1, or a sequence having at least 70%, 75%, 80%, 85%, 90%, 95%, 96%, 97%, 98%, or 99%, or more identity with SEQ ID NO: 1, as described herein. The regulatory element (or regulatory sequence) may comprise or consist of at least one copy of a miR182 target site having a sequence as set forth in SEQ ID NO: 21, or a sequence having at least 70%, 75%, 80%, 85%, 90%, 95%, 96%, 97%, 98%, or 99%, or more identity with SEQ ID NO: 21, as described herein. The regulatory element (or regulatory sequence) may comprise or consist of at least one copy of a miR96 target site having a sequence as set forth in SEQ ID NO: 24, or a sequence having at least 70%, 75%, 80%, 85%, 90%, 95%, 96%, 97%, 98%, or 99%, or more identity with SEQ ID NO: 24, as described herein.

As used herein, the expression “at least one copy” include one, two, three, four, five, six, seven, eight, nine, ten, or more copies of a miR target site of the miR183 family as described herein (such as, for example, of a miR183 target site having a sequence as set forth in SEQ ID NO: 1, or a sequence having at least 70%, 75%, 80%, 85%, 90%, 95%, 96%, 97%, 98%, or 99%, or more identity with SEQ ID NO: 1, as described herein).

In some embodiments, the regulatory element (or regulatory sequence) comprises or consists of one to six copies of the miR target site of the miR183 family as described herein (such as, for example, of the miR183 target site having a sequence as set forth in SEQ ID NO: 1, or a sequence having at least 70%, 75%, 80%, 85%, 90%, 95%, 96%, 97%, 98%, or 99%, or more identity with SEQ ID NO: 1, as described herein). The regulatory element (or regulatory sequence) may thus comprise or consist of one, two, three, four, five, or six copies of the miR target site of the miR183 family as described herein (such as, for example, of the miR183 target site having a sequence as set forth in SEQ ID NO: 1, or a sequence having at least 70%, 75%, 80%, 85%, 90%, 95%, 96%, 97%, 98%, or 99%, or more identity with SEQ ID NO: 1, as described herein).

In some embodiments, the regulatory element (or regulatory sequence) comprises or consists of at least two copies of the miR target site of the miR183 family as described herein (such as, for example, of the miR183 target site having a sequence as set forth in SEQ ID NO: 1, or a sequence having at least 70%, 75%, 80%, 85%, 90%, 95%, 96%, 97%, 98%, or 99%, or more identity with SEQ ID NO: 1, as described herein). In some embodiments, the regulatory element (or regulatory sequence) comprises or consists of two to six copies of the miR target site of the miR183 family as described herein (such as, for example, of the miR183 target site having a sequence as set forth in SEQ ID NO: 1, or a sequence having at least 70%, 75%, 80%, 85%, 90%, 95%, 96%, 97%, 98%, or 99%, or more identity with SEQ ID NO: 1, as described herein).

In some embodiments, the regulatory element (or regulatory sequence) comprises or consists of three copies of the miR target site of the miR183 family as described herein. The regulatory element (or regulatory sequence) may thus comprise or consist of a sequence as set forth in SEQ ID NO: 7, SEQ ID NO: 23, or SEQ ID NO: 26, or a sequence having at least 70%, 75%, 80%, 85%, 90%, 95%, 96%, 97%, 98%, or 99%, or more identity with any one of SEQ ID NO: 7, SEQ ID NO: 23, or SEQ ID NO: 26.

In particular, the regulatory element (or regulatory sequence) may comprise or consist of three copies of the miR183 target site having a sequence as set forth in SEQ ID NO: 1, or a sequence having at least 70%, 75%, 80%, 85%, 90%, 95%, 96%, 97%, 98%, or 99%, or more identity with SEQ ID NO: 1, as described herein. The regulatory element (or regulatory sequence) may thus comprise or consist of a sequence as set forth in SEQ ID NO: 7, or a sequence having at least 70%, 75%, 80%, 85%, 90%, 95%, 96%, 97%, 98%, or 99%, or more identity with SEQ ID NO: 7, as described herein.

In some embodiments, the regulatory element (or regulatory sequence) having a sequence with at least 70%, 75%, 80%, 85%, 90%, 95%, 96%, 97%, 98%, or 99%, or more identity with SEQ ID NO: 7 has a length of at least 300 nucleotides. In some embodiments, the regulatory element (or regulatory sequence) having a sequence with at least 70%, 75%, 80%, 85%, 90%, 95%, 96%, 97%, 98%, or 99%, or more identity with SEQ ID NO: 7 has a length of 300, 305, 310, 315, 320, 325, or 330 nucleotides.

In some embodiments the regulatory element (or regulatory sequence) has a sequence as set forth in SEQ ID NO: 7 with 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, or more nucleotides being substituted by a different nucleotide with reference to the corresponding nucleotide(s) of SEQ ID NO: 7, as described herein. In some embodiments the regulatory element (or regulatory sequence) has a sequence as set forth in SEQ ID NO: 7 with at most 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 nucleotide(s) being substituted by a different nucleotide with reference to the corresponding nucleotide(s) of SEQ ID NO: 7, as described herein.

The regulatory element (or regulatory sequence) may comprise or consist of three copies of the miR182 target site having a sequence as set forth in SEQ ID NO: 21, or a sequence having at least 70%, 75%, 80%, 85%, 90%, 95%, 96%, 97%, 98%, or 99%, or more identity with SEQ ID NO: 21, as described herein. The regulatory element (or regulatory sequence) may thus comprise or consist of a sequence as set forth in SEQ ID NO: 23, or a sequence having at least 70%, 75%, 80%, 85%, 90%, 95%, 96%, 97%, 98%, or 99%, or more identity with SEQ ID NO: 23, as described herein.

In some embodiments, the regulatory element (or regulatory sequence) having a sequence with at least 70%, 75%, 80%, 85%, 90%, 95%, 96%, 97%, 98%, or 99%, or more identity with SEQ ID NO: 23 has a length of at least 300 nucleotides. In some embodiments, the regulatory element (or regulatory sequence) having a sequence with at least 70%, 75%, 80%, 85%, 90%, 95%, 96%, 97%, 98%, or 99%, or more identity with SEQ ID NO: 23 has a length of 300, 305, 310, 315, 320, 325, or 330 nucleotides.

In some embodiments the regulatory element (or regulatory sequence) has a sequence as set forth in SEQ ID NO: 23 with 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, or more nucleotides being substituted by a different nucleotide with reference to the corresponding nucleotide(s) of SEQ ID NO: 23, as described herein. In some embodiments the regulatory element (or regulatory sequence) has a sequence as set forth in SEQ ID NO: 23 with at most 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 nucleotide(s) being substituted by a different nucleotide with reference to the corresponding nucleotide(s) of SEQ ID NO: 23, as described herein.

The regulatory element (or regulatory sequence) may comprise or consist of three copies of the miR96 target site having a sequence as set forth in SEQ ID NO: 24, or a sequence having at least 70%, 75%, 80%, 85%, 90%, 95%, 96%, 97%, 98%, or 99%, or more identity with SEQ ID NO: 24, as described herein. The regulatory element (or regulatory sequence) may thus comprise or consist of a sequence as set forth in SEQ ID NO: 26, or a sequence having at least 70%, 75%, 80%, 85%, 90%, 95%, 96%, 97%, 98%, or 99%, or more identity with SEQ ID NO: 26, as described herein.

In some embodiments, the regulatory element (or regulatory sequence) having a sequence with at least 70%, 75%, 80%, 85%, 90%, 95%, 96%, 97%, 98%, or 99%, or more identity with SEQ ID NO: 26 has a length of at least 210 nucleotides. In some embodiments, the regulatory element (or regulatory sequence) having a sequence with at least 70%, 75%, 80%, 85%, 90%, 95%, 96%, 97%, 98%, or 99%, or more identity with SEQ ID NO: 26 has a length of 200, 205, 210, 215, 220, 225, 230, or 234 nucleotides.

In some embodiments the regulatory element (or regulatory sequence) has a sequence as set forth in SEQ ID NO: 26 with 1, 2, 3, 4, 5, 6, 7, 8, 9, 10, 11, 12, 13, 14, 15, 16, 17, 18, 19, or 20, or more nucleotides being substituted by a different nucleotide with reference to the corresponding nucleotide(s) of SEQ ID NO: 26, as described herein. In some embodiments the regulatory element (or regulatory sequence) has a sequence as set forth in SEQ ID NO: 26 with at most 20, 19, 18, 17, 16, 15, 14, 13, 12, 11, 10, 9, 8, 7, 6, 5, 4, 3, 2, or 1 nucleotide(s) being substituted by a different nucleotide with reference to the corresponding nucleotide(s) of SEQ ID NO: 26, as described herein.

In some embodiments, in the regulatory element (or regulatory sequence) as described herein, comprising several copies of the miR target site of the miR183 family as described herein (such as, for example of the miR183 target site), said copies are separated by a spacer as described herein. In some embodiments, the spacer has a sequence as set forth in SEQ ID NO: 14.

In some embodiments, the regulatory element (or regulatory sequence) as described herein further comprises at least one copy of another miR target site. In some embodiments, the regulatory element (or regulatory sequence) thus comprises or consists of:
at least one copy of a miR183 target site having a sequence as set forth in SEQ ID NO: 1, or a sequence having at least 70%, 75%, 80%, 85%, 90%, 95%, 96%, 97%, 98%, or 99%, or more identity with SEQ ID NO: 1, as described herein; or at least one copy of a miR182 target site having a sequence as set forth in SEQ ID NO: 21, or a sequence having at least 70%, 75%, 80%, 85%, 90%, 95%, 96%, 97%, 98%, or 99%, or more identity with SEQ ID NO: 21, as described herein; or at least one copy of a miR96 target site having a sequence as set forth in SEQ ID NO: 24, or a sequence having at least 70%, 75%, 80%, 85%, 90%, 95%, 96%, 97%, 98%, or 99%, or more identity with SEQ ID NO: 24, as described herein; and
at least one copy of another miR target site.

For example, the other miR target site may be a miR target site recognized by a miRNA expressed in sensory neurons and/or hair cells, such as hair cells of the inner ear. The other miR target may be a miR182 target site, a miR96 target site, a miR194 target site, a miR140 target site, a miR18a target site, a miR99a target site, a miR30b target site, a miR15a target site, or a miR210 target site. In some embodiments, the other miR target site is a miR182 target site.

The miR182 target site may have a sequence complementary to a mature miR182, for example a sequence as set forth in SEQ ID NO: 10, in SEQ ID NO: 12, or a sequence having at least 70%, 75%, 80%, 85%, 90%, 95%, 96%, 97%, 98%, or 99%, or more identity with SEQ ID NO: 10 or SEQ ID NO: 12.

In some embodiments, the regulatory element (or regulatory sequence) as described herein further comprises at least one copy of another miR target site of the miR183 family as described herein. For example, the regulatory element (or regulatory sequence) as described herein may comprise or consist of:
at least one copy of a miR183 target site having a sequence as set forth in SEQ ID NO: 1, or a sequence having at least 70%, 75%, 80%, 85%, 90%, 95%, 96%, 97%, 98%, or 99%, or more identity with SEQ ID NO: 1, as described herein; and
at least one copy of a miR182 target site having a sequence as set forth in SEQ ID NO: 21, or a sequence having at least 70%, 75%, 80%, 85%, 90%, 95%, 96%, 97%, 98%, or 99%, or more identity with SEQ ID NO: 21, as described herein, and/or at least one copy of a miR96 target site having a sequence as set forth in SEQ ID NO: 24, or a sequence having at least 70%, 75%, 80%, 85%, 90%, 95%, 96%, 97%, 98%, or 99%, or more identity with SEQ ID NO: 24, as described herein.

Alternatively, the regulatory element (or regulatory sequence) as described herein may comprise or consist of at least one copy of a miR182 target site having a sequence as set forth in SEQ ID NO: 21, or a sequence having at least 70%, 75%, 80%, 85%, 90%, 95%, 96%, 97%, 98%, or 99%, or more identity with SEQ ID NO: 21, as described herein, and at least one copy of a miR96 target site having a sequence as set forth in SEQ ID NO: 24, or a sequence having at least 70%, 75%, 80%, 85%, 90%, 95%, 96%, 97%, 98%, or 99%, or more identity with SEQ ID NO: 24, as described herein.

As used herein, the expression “nucleic acid sequence of interest” is meant to refer to a nucleic acid sequence to be expressed. In particular, the expression “nucleic acid sequence of interest” is meant to refer to a nucleic acid sequence to be expressed in a controlled manner, for example upon introduction in a host cell or in a host organism. The nucleic acid sequence of interest may be a gene of interest, that is to say a nucleic acid sequence encoding for a functional nucleic acid (such as an RNA) or for a polypeptide or protein.

For example, the nucleic acid of interest, such as the gene of interest, may include an open reading frame encoding for a polypeptide or protein. The nucleic acid sequence of interest may thus include a coding sequence of interest. Alternatively, the nucleic acid sequence of interest may encode for a functional nucleic acid, such as a non-coding RNA.

In some embodiments, the nucleic acid of interest is a gene of interest. As used herein, the term gene may refer in particular to an encoding nucleic acid sequence comprising a coding sequence (or CDS) and at least one regulatory element that is transcribed but not translated, such as a 3′-UTR, a 5′-UTR and/or an intron.

In the expression cassette, the regulatory element may be operably linked to the nucleic acid of interest or inserted in the nucleic acid of interest. Preferably, the regulatory element is inserted in an untranslated region of the nucleic acid of interest. Untranslated regions include for example the 5′-UTR, the 3′-UTR and introns. In some embodiments, the regulatory element is inserted in the 3′-UTR of the nucleic acid of interest, in particular in the 3′-UTR of a gene of interest.

As indicated above, the expression cassette comprises a promoter. For example, the promoter may be a ubiquitous and/or constitutive promoter. Promoters are well known to the skilled artisan, who will know how to select a suitable promoter depending on the nucleic acid sequence of interest to be expressed and on the cells in which expression is desired.

In the expression cassette, the promoter is usually inserted upstream of the nucleic acid sequence of interest to be expressed, that is to say in 5′ of the nucleic acid sequence of interest to be expressed.

The expression cassette may further comprise any sequences or elements that may be required for the expression of the nucleic acid sequence of interest. Such sequences or elements may include, for example, a poly(A) signal, a poly(A) site, an enhancer sequence, a terminator sequence, a degron, a silencer, an insulator and/or an operator.

Another aspect of the present invention is a vector comprising a regulatory element (or regulatory sequence) as described herein, or an isolated nucleic acid coding sequence as described herein, or an expression cassette as described herein.

In particular, the present invention relates to a vector comprising a regulatory element comprising or consisting of at least one copy of a miR target site of the miR183 family, said miR target site having a sequence as set forth in SEQ ID NO: 1, SEQ ID NO: 21, or SEQ ID NO: 24, or a sequence having at least 70%, 75%, 80%, 85%, 90%, 95%, 96%, 97%, 98%, or 99%, or more identity with any one of SEQ ID NO: 1, SEQ ID NO: 21, or SEQ ID NO: 24. For example, the vector may comprise a regulatory element comprising or consisting of at least one copy of a miR183 target site having a sequence as set forth in SEQ ID NO: 1, or a sequence having at least 70%, 75%, 80%, 85%, 90%, 95%, 96%, 97%, 98%, or 99%, or more identity with SEQ ID NO: 1, as described herein.

In some embodiments, the vector comprising a regulatory element as described herein also comprises a nucleic acid sequence of interest as described herein, such as a gene of interest. In such a vector, the regulatory element may be operably linked to the nucleic acid of interest or may be inserted in the nucleic acid of interest. Preferably, the regulatory element is inserted in an untranslated region of the nucleic acid of interest, such as the 5′-UTR, the 3′-UTR or an intron. In some embodiments, the regulatory element is inserted in the 3′-UTR of the nucleic acid of interest, in particular in the 3′-UTR of a gene of interest.

Vectors are well known to the skilled artisan, who will know how to select a suitable vector depending on the nucleic acid sequence of interest to be expressed (for example depending on the length of the nucleic acid sequence of interest) and on the cell in which expression is desired.

Example of vectors include cosmids, plasmids, episomes, artificial chromosomes, phages and viruses (such as lentiviruses, retroviruses, adenoviruses, and adeno-associated viruses (AAVs)), liposomes, lipid nanoparticles, niosomes, polymer-based nanoparticles.

In some embodiments, the vector is an expression vector.

In some embodiments, the vector is one of a liposome, lipid nanoparticle, niosome, or polymer-based nanoparticle, and the vector comprises an isolated nucleic acid coding sequence, preferably an RNA coding sequence, as described herein.

In some embodiments, the vector, in particular the expression vector, is a plasmid. In some embodiments, the vector, in particular the expression vector, is a virus, for example an adeno-associated virus (AAV). Methods for producing a viral vector are well known in the art, and include for example transfecting packaging cells and/or using transient transfection with helper plasmids or viruses.

Another aspect of the present invention is a host cell comprising an isolated sequence as described herein, a regulatory element as described herein, an isolated nucleic acid coding sequence as described herein, an expression cassette as described herein, or a vector as described herein.

In some embodiments, the host cell is an isolated host cell.

In some embodiments, the host cell may be a prokaryote cell, or a eukaryote cell, such as, for example, a yeast cell or an animal cell, in particular a mammalian cell.

It should be noted that, with regards to animal and human cells, the term “host cell” generally refers to a cell of a cultured cell line. Animals and human beings into whom an isolated sequence as described herein, a regulatory element as described herein, an isolated nucleic acid coding sequence as described herein, an expression cassette as described herein, or a vector as described herein has been introduced are explicitly excluded from the definition of a “host cell”.

Another aspect of the present invention is a composition comprising, consisting essentially of, or consisting of an isolated sequence as described herein, an isolated nucleic acid coding sequence as described herein, a regulatory element as described herein, an expression cassette as described herein, a vector as described herein, or a host cell as described herein.

Another aspect of the present invention is a pharmaceutical composition comprising, consisting essentially of, or consisting of an isolated sequence as described herein, an isolated nucleic acid coding sequence as described herein, a regulatory element as described herein, an expression cassette as described herein, a vector as described herein, or a host cell as described herein, and a pharmaceutically acceptable excipient or carrier.

Pharmaceutically acceptable excipients or carriers that may be used in the pharmaceutical composition as described herein include, for example, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol and wool fat.

Another aspect of the present invention is a medicament comprising, consisting essentially of, or consisting of an isolated sequence as described herein, an isolated nucleic acid coding sequence as described herein, a regulatory element as described herein, an expression cassette as described herein, a vector as described herein, or a host cell as described herein.

As used herein “consisting essentially of”, with reference to a composition, pharmaceutical composition, or medicament, means that the isolated nucleic acid sequence as described herein, the isolated nucleic acid coding sequence as described herein, the regulatory element as described herein, the expression cassette as described herein, the vector as described herein, or the host cell as described herein is the only therapeutic agent or agent with a biological activity within said composition, pharmaceutical composition, or medicament.

Another aspect of the present invention is a kit comprising or consisting of an isolated sequence as described herein, a regulatory element as described herein, an isolated nucleic acid coding sequence as described herein, an expression cassette as described herein, a vector as described herein, or a host cell as described herein, and, optionally, instructions for use.

By “kit” is intended any manufacture (e.g., a package or a container) comprising an isolated sequence as described herein, a regulatory element as described herein, an isolated nucleic acid coding sequence as described herein, an expression cassette as described herein, a vector as described herein, or a host cell as described herein. The kit may be promoted, distributed, or sold as a unit for performing the uses or methods as described herein.

Another aspect of the present invention is an isolated sequence as described herein, an isolated nucleic acid coding sequence as described herein, a regulatory element as described herein, an expression cassette as described herein, a vector as described herein, a host cell as described herein, a composition as described herein, or a pharmaceutical composition as described herein for use as a medicament.

Another aspect of the present invention is an isolated sequence as described herein, a regulatory element as described herein, an isolated nucleic acid coding sequence as described herein, an expression cassette as described herein, a vector as described herein, a host cell as described herein, a composition as described herein, a pharmaceutical composition, or a medicament as described herein for use in gene therapy.

Another aspect of the present invention is an isolated sequence as described herein, an isolated nucleic acid coding sequence as described herein, a regulatory element as described herein, an expression cassette as described herein, a vector as described herein, a host cell as described herein, a composition as described herein, a pharmaceutical composition, or a medicament as described herein for use in the treatment of sensory impairment.

Another aspect of the present invention is a pharmaceutical composition as described herein for the treatment of a sensory impairment or for use in the treatment of a sensory impairment.

Another aspect of the present invention is the use of an isolated sequence as described herein, an isolated nucleic acid coding sequence as described herein, a regulatory element as described herein, an expression cassette as described herein, a vector as described herein, or a host cell as described herein, for the manufacture of a medicament for the treatment of a sensory impairment.

Another aspect of the present invention is a method of gene therapy in a subject in need thereof, said method comprising administering to the subject an isolated sequence as described herein, an isolated nucleic acid coding sequence as described herein, a regulatory element as described herein, an expression cassette as described herein, or a vector as described herein.

Another aspect of the present invention is a method for treating a sensory impairment in a subject in need thereof, said method comprising administering to the subject an isolated sequence as described herein, an isolated nucleic acid coding sequence as described herein, a regulatory element as described herein, an expression cassette as described herein, or a vector as described herein.

In some embodiments, the sensory impairment is a hereditary or congenital sensory impairment, that is to say a sensory impairment of genetic origin.

Another aspect of the present invention is the use, in particular the in vitro or ex vivo use, of an isolated sequence as described herein, an isolated nucleic acid coding sequence as described herein, or an expression cassette as described herein for producing a vector, in particular for producing an expression vector.

Another aspect of the present invention is the use, in particular the in vitro or ex vivo use, of a vector as described herein for producing a host cell as described herein.

Another aspect of the present invention is the use, in particular the in vitro or ex vivo use, of an isolated sequence as described herein, an isolated nucleic acid coding sequence as described herein, a regulatory element as described herein, an expression cassette as described herein, or a vector as described herein for specifically expressing a gene of interest in cells not expressing a miRNA of the miR183 family, that is to say in cells not expressing miR183, miR182 and/or miR96. For example, an isolated sequence as described herein, an isolated nucleic acid coding sequence as described herein, a regulatory element as described herein, an expression cassette as described herein, or a vector as described herein comprising a miR183 target site as described herein may be used in particular for specifically expressing a gene of interest in cells not expressing a miR183. Similarly, an isolated sequence as described herein, an isolated nucleic acid coding sequence as described herein, a regulatory element as described herein, an expression cassette as described herein, or a vector as described herein comprising a miR182 target site as described herein may be used in particular for specifically expressing a gene of interest in cells not expressing a miR182; and an isolated sequence as described herein, an isolated nucleic acid coding sequence as described herein, a regulatory element as described herein, an expression cassette as described herein, or a vector as described herein comprising a miR96 target site as described herein may be used in particular for specifically expressing a gene of interest in cells not expressing a miR96.

Another aspect of the present invention is the use, in particular the in vitro or ex vivo use, of an isolated sequence as described herein, an isolated nucleic acid coding sequence as described herein, a regulatory element as described herein, an expression cassette as described herein, or a vector as described herein for modulating or controlling the expression of a nucleic acid of interest, such as a gene of interest.

Another aspect of the present invention is the use of an isolated sequence as described herein, an isolated nucleic acid coding sequence as described herein, a regulatory element as described herein, an expression cassette as described herein, or a vector as described herein for reducing the off-target expression of a nucleic acid of interest, such as a gene of interest, wherein expression of the nucleic acid of interest is to be prevented (or repressed or inhibited) in cells and/or tissues expressing at least one miRNA of the miR183 family, that is to say in cells and/or tissues expressing miR183, miR182 and/or miR96.

Another aspect of the present invention is a method for producing a vector as described herein, said method comprising inserting in a vector the regulatory element as described herein.

Another aspect of the present invention is a method, in particular an in vitro or ex vivo method, for modulating or controlling the expression of a nucleic acid of interest, such as a gene of interest, in a tissue comprising at least two different cell types, said method comprising introducing in said tissue an isolated nucleic acid coding sequence as described herein, a regulatory element as described herein, an expression cassette as described herein, or a vector as described herein.

Another aspect of the present invention is a method for modulating or controlling the expression of a nucleic acid of interest, such as a gene of interest, in a subject in need of gene therapy, said method comprising introducing in a tissue of the subject an isolated nucleic acid coding sequence as described herein, a regulatory element as described herein, an expression cassette as described herein, or a vector as described herein, wherein said tissue comprises at least two different cell types.

A subject in need of gene therapy may be a subject suffering from a sensory impairment, in particular from a congenital a sensory impairment.

As used herein, the expression “introducing in a tissue of the subject” may correspond to a systemic administration to the subject or to a local administration to the subject.

In particular, the tissue comprising at least two different cell types may comprise at least one cell type in which expression of the nucleic acid of interest is desired and at least one cell type in which expression of the nucleic acid of interest is not desired.

In some embodiments, the tissue comprising at least two different cell types comprises at least one type of cells expressing at least one miRNA of the miR183 family (i.e., miR183, miR182 and/or miR96) and at least one type of cells not expressing any miRNA of the miR183 family. In particular, the tissue comprising at least two different cell types may comprise at least one type of cells expressing miR183 and at least one type of cells not expressing miR183. The tissue comprising at least two different cell types may comprise at least one type of cells expressing miR182 and at least one type of cells not expressing miR182. The tissue comprising at least two different cell types may comprise at least one type of cells expressing miR96 and at least one type of cells not expressing miR96.

As used herein, by “not expressing any miRNA of the miR183 family” it is meant not expressing any miRNA of the miR183 in an amount sufficient for the miRNA to exert a biological activity, in particular in an amount sufficient to mediate gene silencing.

Another aspect of the present invention is a method for reducing the off-target expression of a nucleic acid of interest, such as a gene of interest, wherein expression of the nucleic acid of interest is to be prevented (or repressed or inhibited) in cells and/or tissues expressing at least one miRNA of the miR183 family, that is to say in cells and/or tissues expressing miR183, miR182 and/or miR96, said method comprising introducing in a tissue (for example in a tissue of a subject in need of gene therapy) an isolated nucleic acid coding sequence as described herein, a regulatory element as described herein, an expression cassette as described herein, or a vector as described herein, wherein said tissue comprises at least two different cell types as described herein.

TABLE OF SEQUENCES

| SEQ | | |
|---|---|---|
| ID | Sequence | |
| NO | function | Sequence |
| 1 | precursor miR183 | TCGTGGATCTGTCTCTGCTCTGTTTATGGCCCTTCGGTA |
| | target site (DNA | ATTCACTGACTGAGACTGTTCACAGTGAATTCTACCAG |
| | or RNA) | TGCCATACACAGAACAGGAGTCACACTCTGCGG |
| 2 | human precursor | CCGCAGAGTGTGACTCCTGTTCTGTGTATGGCACTGGT |
| | miR183 (RNA) | AGAATTCACTGTGAACAGTCTCAGTCAGTGAATTACCG |
| | | AAGGGCCATAAACAGAGCAGAGACAGATCCACGA |
| 3 | hsa-miR183-5p | TATGGCACTGGTAGAATTCACT |
| | (RNA) | |
| 4 | hsa-miR183-3p | GTGAATTACCGAAGGGCCATAA |
| | (RNA) | |
| 5 | miR183-5p target | AGTGAATTCTACCAGTGCCATA |
| | (DNA or RNA) | |
| 6 | miR183-3p target | TTATGGCCCTTCGGTAATTCAC |
| | (DNA or RNA) | |
| 7 | precursor miR183 | TCGTGGATCTGTCTCTGCTCTGTTTATGGCCCTTCGGTA |
| | target site- | ATTCACTGACTGAGACTGTTCACAGTGAATTCTACCAG |
| | 3 copies (DNA or | TGCCATACACAGAACAGGAGTCACACTCTGCGGTCGT |
| | RNA) | GGATCTGTCTCTGCTCTGTTTATGGCCCTTCGGTAATTC |
| | | ACTGACTGAGACTGTTCACAGTGAATTCTACCAGTGCC |
| | | ATACACAGAACAGGAGTCACACTCTGCGGTCGTGGAT |
| | | CTGTCTCTGCTCTGTTTATGGCCCTTCGGTAATTCACTG |
| | | ACTGAGACTGTTCACAGTGAATTCTACCAGTGCCATAC |
| | | ACAGAACAGGAGTCACACTCTGCGG |
| 8 | miR183-5p target | AGTGAATTCTACCAGTGCCATAATAACTAAAAGATTCG |
| | 3 copies (DNA or | GAAGTGAATTCTACCAGTGCCATAATAACTAAAAGATT |
| | RNA) | CGGAAGTGAATTCTACCAGTGCCATA |
| 9 | miR183 5p+3p | AGTGAATTCTACCAGTGCCATAATAACTAAAAGATTCG |
| | target-3 copies | GATTATGGCCCTTCGGTAATTCACATAACTAAAAGATT |
| | each (DNA or | CGGAAGTGAATTCTACCAGTGCCATAATAACTAAAAG |
| | RNA) | ATTCGGATTATGGCCCTTCGGTAATTCACATAACTAAA |
| | | AGATTCGGAAGTGAATTCTACCAGTGCCATAATAACTA |
| | | AAAGATTCGGATTATGGCCCTTCGGTAATTCAC |
| 10 | miR182-5p target | AGTGTGAGTTCTACCATTGCCAAA |
| | (DNA or RNA) | |
| 11 | miR182-5p | TTTGGCAATGGTAGAACTCACACT |
| | (RNA) | |
| 12 | miR182-3p target | TAGTTGGCAAGTCTAGAACCA |
| | (DNA or RNA) | |
| 13 | miR182-3p | TGGTTCTAGACTTGCCAACTA |
| | (RNA) | |
| 14 | spacer (DNA) | ATAACTAAAAGATTCGGA |
| 15 | spacer (DNA) | AATATATATATATTATTA |
| 16 | spacer (DNA) | AAAAACATATAAAATAAT |
| 17 | spacer (DNA) | CTTTCTTTTCCCAATTTT |
| 18 | primer (DNA) | GAGCGCACCATCTTCTTCA |
| 19 | primer (DNA) | TTCAGCTCGATGCGGTTC |
| 20 | Probe (DNA) | AGGACGACGGCAACTACAAGACC |
| 21 | precursor miR182 | GTGCCGGCTGAGTCCTCGCCCCATAGTTGGCAAGTCTA |
| | target site (DNA | GAACCACCGGATCCTGTTACCTCACCAGTGTGAGTTCT |
| | or RNA) | ACCATTGCCAAAAACGGGGGGAGGCAAGCAGCTC |
| 22 | human precursor | GAGCTGCTTGCCTCCCCCCGTTTTTGGCAATGGTAGAA |
| | miR182 (RNA) | CTCACACTGGTGAGGTAACAGGATCCGGTGGTTCTAGA |
| | | CTTGCCAACTATGGGGCGAGGACTCAGCCGGCAC |
| 23 | precursor miR182 | GTGCCGGCTGAGTCCTCGCCCCATAGTTGGCAAGTCTA |
| | target site | GAACCACCGGATCCTGTTACCTCACCAGTGTGAGTTCT |
| | 3 copies (DNA or | ACCATTGCCAAAAACGGGGGGAGGCAAGCAGCTCGTG |
| | RNA) | CCGGCTGAGTCCTCGCCCCATAGTTGGCAAGTCTAGAA |
| | | CCACCGGATCCTGTTACCTCACCAGTGTGAGTTCTACC |
| | | ATTGCCAAAAACGGGGGGAGGCAAGCAGCTCGTGCCG |
| | | GCTGAGTCCTCGCCCCATAGTTGGCAAGTCTAGAACCA |
| | | CCGGATCCTGTTACCTCACCAGTGTGAGTTCTACCATT |
| | | GCCAAAAACGGGGGGAGGCAAGCAGCTC |
| 24 | precursor miR96 | TTTCCCATATTGGCACTGCACATGATTGCTCAGAGCGG |
| | target site (DNA | AGAGACACAAGCAAAAATGTGCTAGTGCCAAAATCGG |
| | or RNA) | CCA |
| 25 | human precursor | TGGCCGATTTTGGCACTAGCACATTTTTGCTTGTGTCTC |
| | miR96 (RNA) | TCCGCTCTGAGCAATCATGTGCAGTGCCAATATGGGAA |
| | | A |
| 26 | precursor miR96 | TTTCCCATATTGGCACTGCACATGATTGCTCAGAGCGG |
| | target site- | AGAGACACAAGCAAAAATGTGCTAGTGCCAAAATCGG |
| | 3 copies (DNA or | CCATTTCCCATATTGGCACTGCACATGATTGCTCAGAG |
| | RNA) | CGGAGAGACACAAGCAAAAATGTGCTAGTGCCAAAAT |
| | | CGGCCATTTCCCATATTGGCACTGCACATGATTGCTCA |
| | | GAGCGGAGAGACACAAGCAAAAATGTGCTAGTGCCAA |
| | | AATCGGCCA |
| 27 | miR96-5p target | AGCAAAAATGTGCTAGTGCCAAA |
| | (DNA or RNA) | |
| 28 | miR96-5p (RNA) | TTTGGCACTAGCACATTTTTGCT |
| 29 | miR96-3p target | CATATTGGCACTGCACATGATT |
| | (DNA or RNA) | |
| 30 | miR96-3p (RNA) | AATCATGTGCAGTGCCAATATG |

### BRIEF DESCRIPTION OF THE DRAWINGS

FIGS. 1A-B are schematic representations of AAV cassettes comprising at least a promoter, a transgene (i.e., GFP) and a polyadenylation or poly(A) signal (pA). When present, a dashed box represents one so-called “precursor miR183 target site” or “precursor miRT183” (a miR183 target site having a sequence as set forth in SEQ ID NO: 1). FIG. 1A represents an AAV-GFP construct which does not comprise any miR183 target site. FIG. 1B represents an AAV-GFP-precursor miRT183 construct comprising 3 copies of the precursor miR183 target site, inserted in 3′ (i.e., downstream) of the GFP encoding sequence.

FIGS. 2A-E are schematic representations of plasmids comprising at least a promoter (i.e., smCBA promoter), a transgene (i.e., GFP) and a polyadenylation or poly(A) signal (pA). When present, a dashed box represents one precursor miR183 target site, which comprises both the sequence complementary to has-miR183-5p (“5p binding site” or 5P) and the sequence complementary hashsa-miR183-3p (“3p binding site or 3P). When present, a black box represents one 5p binding site (5P) and a white box represents one 3p binding site (3P). The smaller hatched box depicts the presence of a spacer (between 5p and/or 3p binding sites). FIG. 2A represents a plasmid construct which does not comprise any miR183 target site (0× precursor miRT183 or no miRT183). FIG. 2B represents a plasmid construct comprising one copy of the precursor miR183 target site, downstream of a smCBA-GFP cassette (1× precursor miRT183). FIG. 2C represents a plasmid construct comprising 3 copies of the precursor miR183 target site, downstream of a smCBA-GFP cassette (3× precursor miRT183). FIG. 2D represents a plasmid construct comprising 3 copies of a miR183 target site consisting of the 5P binding site, downstream of a smCBA-GFP cassette (3× miRT183 5P). The copies are interspaced with a spacer, represented by a smaller hatched box. FIG. 2E represents a plasmid construct comprising 3 copies of a miR183 target site consisting of a tandem of the 5P and 3P binding sites, downstream of a smCBA-GFP cassette (3×miRT183 5+3P). The 5P and 3P binding sites are separated by a spacer, represented by a smaller hatched box.

FIGS. 3A-E illustrate the regulatory effect of the precursor miR183 target site on the expression of a transgene in the inner ear of mature mice. FIGS. 3A-D are confocal microscopy images showing the organ of Corti of mice treated with the AAV-GFP construct as depicted in FIG. 1A (FIGS. 3A-B) or with the AAV-GFP-precursor miRT183 construct as depicted in FIG. 1B (FIGS. 3C-D). FIGS. 3B and D show staining for the hair cells present in the organ of Corti with myosin VIIa (MyoVIIa). FIGS. 3A and C show GFP expression in the organ of Corti. Scale bar is indicated. FIG. 3E is a graph showing the quantification of GFP-positive inner hair cells (IHC) and outer hair cells (OHC) in mice treated with the AAV-GFP construct or with the AAV-GFP-precursor miRT183 construct. Data are shown as mean±SD. Statistical analyses were performed by ANOVA (****=p<0.0001).

FIGS. 4A-D illustrate the regulatory effect of the precursor miR183 target site on the expression of a transgene in HEK293 cells. FIGS. 4A-C are confocal images of HEK293 cells transfected with GFP-expressing plasmids containing either 0 (FIG. 4A), 1 (FIG. 4B) or 3 (FIG. 4C) copy (ies) of the precursor miR183 target site. White dots represent GFP-positive cells. FIG. 4D is a graph showing the quantification of GFP fluorescence measured in cell lysates and normalized versus control (i.e., 0 copy of the precursor miR183 target site).

FIGS. 5A-B are graphs comparing the regulatory effect of the so-called precursor miR183 target site and of the so-called mature miR183 target sites on the expression of a transgene. HEK293 cells were transfected with different plasmid constructs comprising different miR183 target sites as depicted in FIG. 2. FIG. 5A shows plasmid content in cell lysates measured by quantitative polymerase chain reaction (qPCR). FIG. 5B shows GFP fluorescence measured in cell lysates. Statistical analysis was performed using Fisher's LSD multiple comparisons: ns=not significant; *=p<0.05.

FIGS. 6A-B are graphs comparing the regulatory effect of 2 copies and 3 copies of the so-called precursor miR183 target site on the expression of a transgene. HEK293 cells were transfected with plasmids comprising the precursor miR183 target site as indicated (2× precursor miRT183 or 3× precursor miRT183) downstream of a GFP-expressing cassette. The graphs show the quantification of GFP fluorescence measured in cell lysates and normalized versus control (i.e., no miR target site) 24 h after medium replacement (FIG. 6A) or 48h after medium replacement (FIG. 6B). Error bars represent standard error of the mean (SEM). *: p<0.05, **: p<0.01; one-way ANOVA.

FIGS. 7A-B are graphs showing the regulatory effect of the so-called precursor miR182 target site on the expression of a transgene. HEK293 cells were transfected with plasmids comprising 3 copies of the precursor miR182 target site (3× precursor miRT182) downstream of a GFP-expressing cassette. The graphs show the quantification of GFP fluorescence measured in cell lysates and normalized versus control (i.e., no miR target site) 24 h after medium replacement (FIG. 7A) or 48h after medium replacement (FIG. 7B). Error bars represent standard error of the mean. ****: p<0.0001; unpaired two-tailed T Test.

FIGS. 8A-B are graphs showing the regulatory effect of the so-called precursor miR96 target site on the expression of a transgene. HEK293 cells were transfected with plasmids comprising 3 copies of the precursor miR96 target site (3× precursor miRT96) downstream of a GFP-expressing cassette. The graphs show the quantification of GFP fluorescence measured in cell lysates and normalized versus control (i.e., no miR target site) 24 h after medium replacement (FIG. 8A) or 48h after medium replacement (FIG. 8B). Error bars represent standard error of the mean. *: p<0.05, ***: p<0.001; unpaired two-tailed T Test.

### EXAMPLES

The present invention is further illustrated by the following examples.

### Example 1

### Materials and Methods

### Mice and Cell Lines

Wild-type C57Bl/6 mice were kept in standard conditions under: 12h/12h light/dark cycles and environmental enrichment. Studies were conducted in compliance with animal health regulations, in particular: Council Directive No. 2010/63/EU of 22 Sep. 2010 and French decree No. 2013-118 of 1 Feb. 2013 on the protection of animals used for scientific purposes.

HEK293 cells (human embryonic kidney 293 cell) were cultured in DMEM with 10% fetal bovine serum (FBS) in standard conditions.

### Vectors

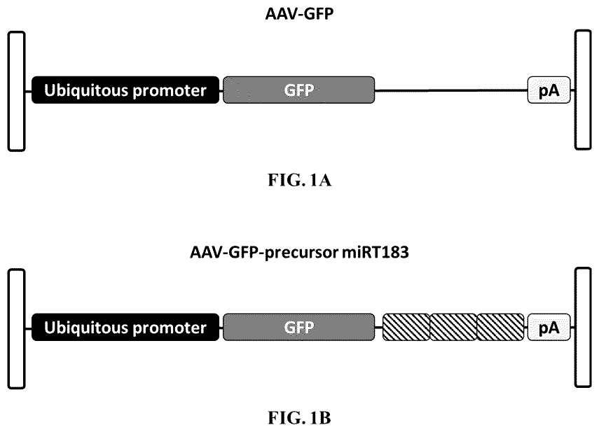

AAV vectors were constructed for administration to mice. The AAV-GFP control construct comprises a GFP-expressing cassette in which the GFP encoding sequence is under the control of a ubiquitous promoter (see FIG. 1A). In the AAV-GFP-precursor miRT183 construct, 3 copies of the miR183 target site having a sequence as set forth in SEQ ID NO: 1 (so-called “precursor miR183 target site”), which is complementary to the human sequence of the precursor miR183 (i.e., SEQ ID NO: 2), were inserted in 3′ (i.e., downstream) of the GFP encoding sequence (see FIG. 1B).

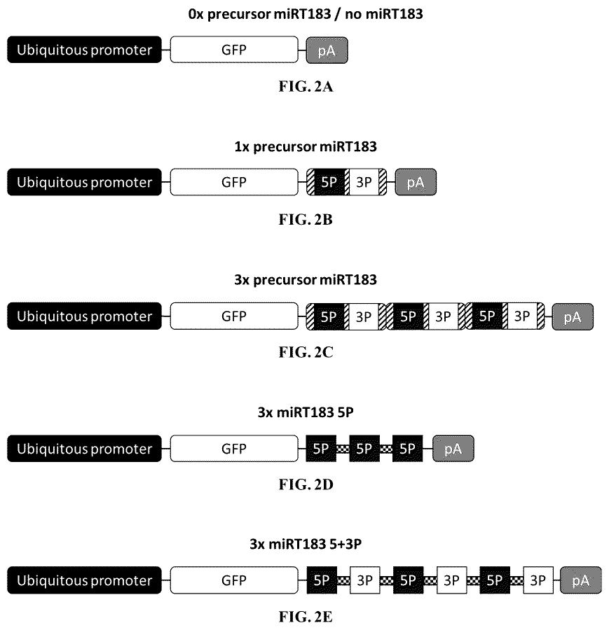

Plasmid vectors were constructed for transfection in HEK293 cells using a pAAV-smCBA-eGFP-bGH backbone (Genscript). As shown on FIG. 2A, the “no miRT183” control construct comprises a GFP-expressing cassette in which the eGFP encoding sequence is under the control of the ubiquitous promoter smCBA (truncated chimeric cytomegalovirus (CMV)-chicken β-actin). Plasmids comprising miR183 target sites were obtained by cloning micro RNA target sites into a pAAV-smCBA-eGFP-bGH backbone (Genscript):
a plasmid comprising one copy of the so-called precursor miR183 target site having a sequence as set forth in SEQ ID NO: 1, downstream of a smCBA-eGFP cassette (1× precursor miRT183—FIG. 2B);
a plasmid comprising 3 copies of the so-called precursor miR183 target site having a sequence as set forth in SEQ ID NO: 1, downstream of a smCBA-eGFP cassette (3× precursor miRT183—FIG. 2C);
a plasmid comprising 3 copies of a miR183 target site consisting of the sequence as set forth in SEQ ID NO: 5 (so-called “5p binding site”), which is complementary to hsa-miR183-5p (i.e., SEQ ID NO: 3), downstream of a smCBA-GFP cassette (3×miRT183 5P—FIG. 2D); the 3 copies of the so-called “5p binding site” are interspaced with a spacer having a sequence as set forth in SEQ ID NO: 14; and
a plasmid comprising 3 copies of a miR183 target site consisting of a tandem of the sequence as set forth in SEQ ID NO: 5 (so-called “5p binding site” which is complementary to hsa-miR183-5p (i.e., SEQ ID NO: 3)) and of the sequence as set forth in SEQ ID NO: 6 (so-called “3p binding site” which is complementary to hsa-miR183-3p (i.e., SEQ ID NO: 4)), downstream of a smCBA-GFP cassette (3×miRT183 5+3P—FIG. 2E); the so-called “5p binding site” and “3p binding site” are separated by a spacer having a sequence as set forth in SEQ ID NO: 14.
   In the plasmid “3×miRT183 5P” of FIG. 2D, the regulatory element comprising three copies of the miR183 target site consisting of the sequence as set forth in SEQ ID NO: 5 (so-called “5p binding site”) and two spacers has a sequence as set forth in SEQ ID NO: 8. In the plasmid “3×miRT183 5+3P” of FIG. 2E, the regulatory element comprising three copies of the miR183 target site consisting of a tandem of the sequence as set forth in SEQ ID NO: 5 (so-called “5p binding site”) and of the sequence as set forth in SEQ ID NO: 6 (so-called “3p binding site”) and five spacers has a sequence as set forth in SEQ ID NO: 9.

### In Vivo Experiments

At postnatal day 14 (P14), wild-type C57Bl/6 mice were administered with adeno-associated viral vectors (AAV) comprising the nucleotide sequence encoding GFP (green fluorescent protein). A total volume of 1 μl of AAV vector was injected into the mice inner ear through the round window membrane. As indicated in FIG. 1, either the AAV-GFP construct or the AAV-GFP-precursor miRT183 construct were injected, at a concentration of 5×10¹³ vg/mL (vector genome per mL). Two weeks after injection, the mice were euthanized, temporal bones were collected and fixed for histological analysis. After dissection of the cochleae, organs of Corti were immunolabeled with myosin VIIa (MyoVIIa). In the cochleae, myosin VIIa is specifically expressed in hair cells. Organs of Corti were then analyzed by confocal microscopy to assess the expression of both MyoVIIa and GFP.

### In Vitro Experiments

To assess the effect on gene expression of regulatory elements comprising miR183 target site(s), several plasmids were synthesized and compared (see FIG. 2). Each plasmid (20 μg) was transfected in HEK293 cells in T75 flasks using polyethylenimine (PEI) diluted in OptiMEM culture medium. After 7 hours, PEI-DNA-OptiMEM medium was replaced by DMEM 2% FBS. Three days after transfection, cells were collected, PBS-washed and lysed with PBS-Tween 20 (0.2%). After clarification of the lysate by centrifugation, GFP fluorescence was measured using the Synergy plate reader (Agilent) and results provided as arbitrary units. In parallel, protein content was determined using the BCA assay kit (ThermoFisher). Plasmid copy number in cell lysates was measured by TaqMan qPCR using GFP primers and probe as follows:

eGFP fwd:
(SEQ ID NO: 18)
5′-GAGCGCACCATCTTCTTCA;
eGFP rev:
(SEQ ID NO: 19)
5′-TTCAGCTCGATGCGGTTC;
and
eGFP probe:
(SEQ ID NO: 20)
5′-AGGACGACGGCAACTACAAGACC.

### Results

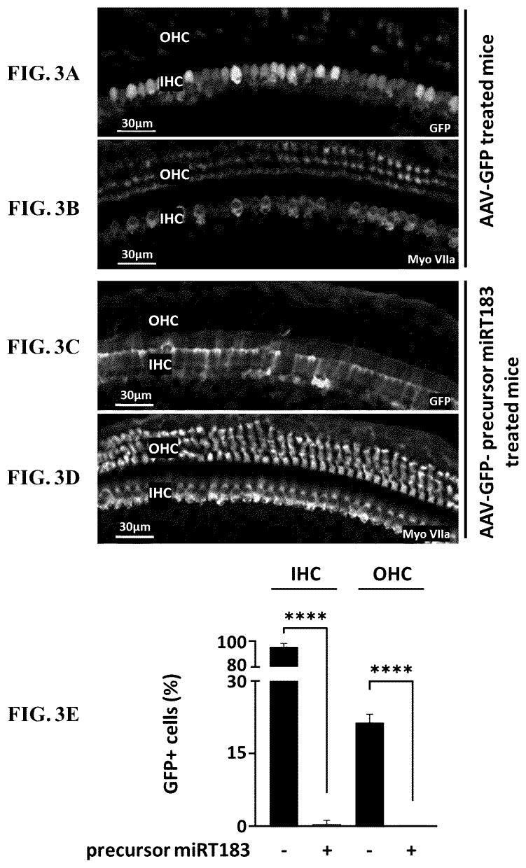

In order to evaluate the regulatory effect of the so-called precursor miR183 target site (i.e., the miR183 target site having a sequence as set forth in SEQ ID NO: 1) on the expression of a transgene, mice were injected with an AAV-GFP-precursor miRT183 construct comprising a regulatory element consisting of 3 copies of the precursor miR183 target site (see FIG. 1A). As a control, mice were injected with an AAV-GFP construct not comprising any regulatory element (see FIG. 1B). Two weeks after injection, mice were euthanized, their cochleae were collected, stained for myosin VIIA and GFP and analyzed by confocal microscopy. Analysis of cochleae from mice injected with the AAV-GFP control construct revealed GFP and MyoVIIA colocalization in the inner hair cell layer (FIGS. 3A-B), showing efficient GFP expression in the hair cells (inner hair cells, IHC and outer hair cells, OHC). By contrast, the presence of a regulatory element comprising 3 copies of the precursor miR183 target site inhibited GFP expression in the inner hair cells of cochleae from mice injected with the AAV-GFP-precursor miR183 construct, as demonstrated by the absence of colocalization between GFP and MyoVIIa and remaining GFP signal persisting in underlying support cells (FIGS. 3C-D). The regulatory effect of the precursor miR183 target site was also demonstrated following quantification of GFP-positive hair cells from cochleae of mice injected with the AAV-GFP construct or with the AAV-GFP-precursor miRT183 construct. As shown on FIG. 3E, the proportion of GFP-positive cells was significantly higher for both inner hair cells (IHC) and outer hair cells (OHC) of mice injected with the AAV-GFP construct, that is to say in the absence of the regulatory element comprising the miR183 target site (FIG. 3E).

These data demonstrate that the presence of the so-called precursor miR183 target site having a sequence as set forth in SEQ ID NO: 1, downstream of the GFP-expressing cassette, significantly inhibited GFP expression in the hair cells of mice treated with the AAV-GFP-precursor miRT183.

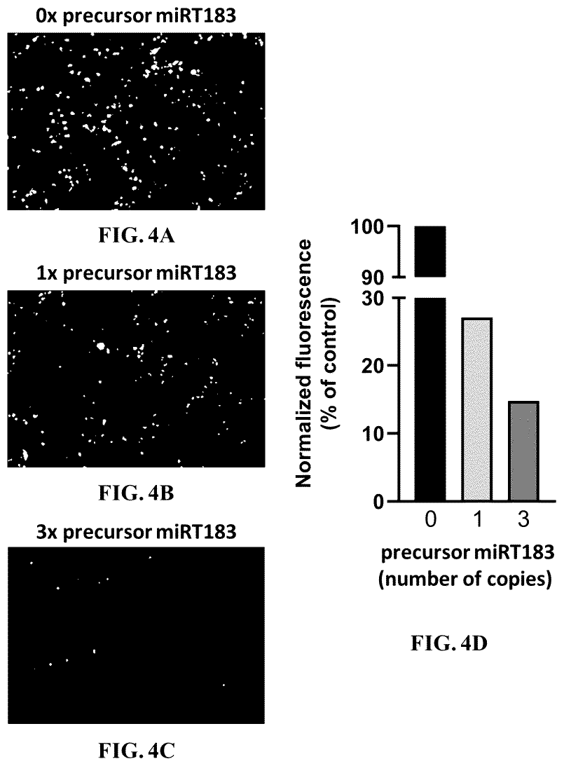

To further evaluate the regulatory effect of the precursor miR183 target site on the expression of a transgene, HEK293 cells were transfected with plasmid constructs containing either no miR183 target site (as described on FIG. 2A), 1 copy of the precursor miR183 target site (as described on FIG. 2B) or 3 copies of the precursor target site (as described on FIG. 2C), downstream of a GFP-expressing cassette. GFP expression in the transfected cells was assessed using a fluorescence plate reader after lysis of the cells. As shown on FIGS. 4A-C, increasing number of copies of the precursor miR183 target site in the plasmid construct resulted in a decrease in the number of GFP-positive cells. FIG. 4D shows the quantification of GFP fluorescence in transfected HEK293 cell lysate, and confirms (i) that the presence of one precursor miR183 target site was sufficient to strongly inhibit GFP expression, and (ii) that increasing the number of precursor miR183 target sites strengthened the inhibition of GFP expression.

These data demonstrate that efficient repression of a transgene expression can be obtained with vectors comprising a regulatory element consisting of one so-called precursor miR183 target site having a sequence as set forth in SEQ ID NO: 1. Additionally, these data demonstrate that a vector comprising a regulatory element comprising several copies of the precursor miR183 target site displays an even stronger inhibitory effect on the transgene expression.

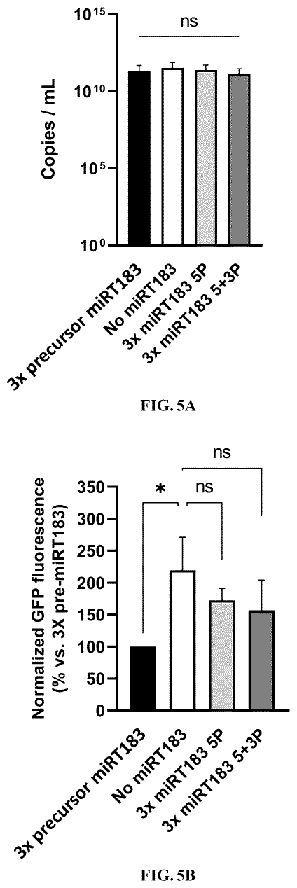

In order to compare the regulatory effect of the precursor miR183 target site with that of a so-called “mature miR183 target site” consisting of a sequence complementary to the sequence of a mature miR183, HEK293 cells were transfected with plasmid constructs comprising either no miR183 target site (as described on FIG. 2A), 3 copies of the precursor miR183 target site (as described on FIG. 2C), 3 copies of a so-called “mature miR183 target site” consisting of the sequence as set forth in SEQ ID NO: 5 (so-called “5p binding site”), which is complementary to hsa-miR183-5p (as described on FIG. 2D), or 3 copies of a so-called “mature miR183 target site” consisting of a tandem of the sequence as set forth in SEQ ID NO: 5 (so-called “5p binding site”) which is complementary to hsa-miR183-5p) and of the sequence as set forth in SEQ ID NO: 6 (so-called “3p binding site”) which is complementary to hsa-miR183-3p (as described on FIG. 2E), downstream of a GFP-expressing cassette. First, transfection efficacy was assessed by quantitative PCR, and showed that a similar transfection efficacy was obtained with each of the four plasmid constructs (FIG. 5A). Then, GFP fluorescence was measured in cell lysates using a fluorescence plate reader and revealed a significant reduction of GFP expression in cells transfected with the plasmid comprising 3 copies of the precursor miR183 target site, as compared to the GFP expression in cells transfected with the control construct without any miR183 target site (FIG. 5B). Strikingly, both plasmids comprising 3 copies of a so-called “mature miR183 target site” failed to induce a significant reduction of GFP expression, as compared to the GFP expression in cells transfected with the control construct without any miR183 target site (FIG. 5B).

These data unexpectedly demonstrate a superior effect in transgene silencing of the precursor miR183 target site having a sequence as set forth in SEQ ID NO: 1 that is complementary to the sequence of the precursor miR183, as compared to the so-called “mature miR183 target sites” having a sequence complementary to the sequence of a mature miR183.

### Example 2

### Materials and Methods

### Vectors

Plasmid vectors were constructed for transfection in HEK293 cells using a pAAV-smCBA-eGFP-bGH backbone (Genscript). As indicated in Example 1, the “no miRT” control construct comprises a GFP-expressing cassette in which the eGFP encoding sequence is under the control of the ubiquitous promoter smCBA (truncated chimeric cytomegalovirus (CMV)-chicken β-actin). Plasmids comprising miRNA target sites were obtained by cloning micro RNA target sites into a pAAV-smCBA-eGFP-bGH backbone (Genscript):
a plasmid comprising 2 copies of the so-called precursor miR183 target site having a sequence as set forth in SEQ ID NO: 1, downstream of a smCBA-eGFP cassette (3× precursor miRT183);
a plasmid comprising 3 copies of the so-called precursor miR183 target site having a sequence as set forth in SEQ ID NO: 1, downstream of a smCBA-eGFP cassette;
a plasmid comprising 3 copies of the so-called precursor miR182 target site having a sequence as set forth in SEQ ID NO: 21, which is complementary to the human sequence of the precursor miR182 (i.e., SEQ ID NO: 22), downstream of a smCBA-eGFP cassette (3× precursor miRT182);
a plasmid comprising 3 copies of the so-called precursor miR96 target site having a sequence as set forth in SEQ ID NO: 24, which is complementary to the human sequence of the precursor miR182 (i.e., SEQ ID NO: 25), downstream of a smCBA-eGFP cassette.

### In Vitro Experiments

To confirm the effect on gene expression of regulatory elements comprising miR target sites of the miR183 family as described herein, several plasmids were synthesized. Each plasmid (2 μg) was transfected to HEK293 cells in 6-well plate using polyethylenimine (PEI) diluted in OptiMEM culture medium. After 12 hours, PEI-DNA-OptiMEM medium was replaced by DMEM 2% FBS (fetal bovine serum). Two or three days after transfection (24h or 48h after medium replacement), cells were collected, PBS-washed and lysed with PBS-Tween20 (0.2%). After clarification of the lysate by centrifugation, GFP fluorescence was measured using the Synergy plate reader (Agilent). In parallel, protein content was determined using the BCA assay kit (ThermoFisher) and used to normalize the data.

### Results

HEK293 cells were transfected with plasmid constructs containing either no miR target site, 2 copies of the precursor miR183 target site, 3 copies of the precursor miR183 target site (corresponding to SEQ ID NO: 7), 3 copies of the precursor miR182 target site (corresponding to SEQ ID NO: 23), or 3 copies of the precursor miR96 target site (corresponding to SEQ ID NO: 26), downstream of a GFP-expressing cassette. GFP expression in the transfected cells was assessed using a fluorescence plate reader after lysis of the cells, either 24 h after medium replacement (i.e., two days after transfection) or 48h after medium replacement (i.e., three days after transfection).

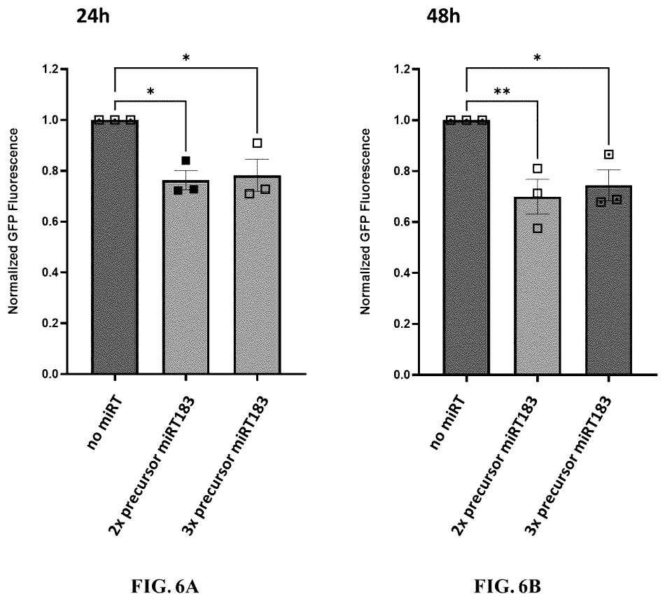

FIG. 6 shows the quantification of GFP fluorescence in transfected HEK293 cell lysate, and confirms that the presence of the precursor miR183 target site significantly inhibits GFP expression. Of note, the inhibition of GFP expression is maintained over time, with similar levels of inhibition observed 24 h after medium replacement (FIG. 6A) and 48h after medium replacement (FIG. 6B). FIG. 6 also confirms that the presence of 2 copies of the precursor miR183 target site is sufficient to significantly inhibit GFP expression.

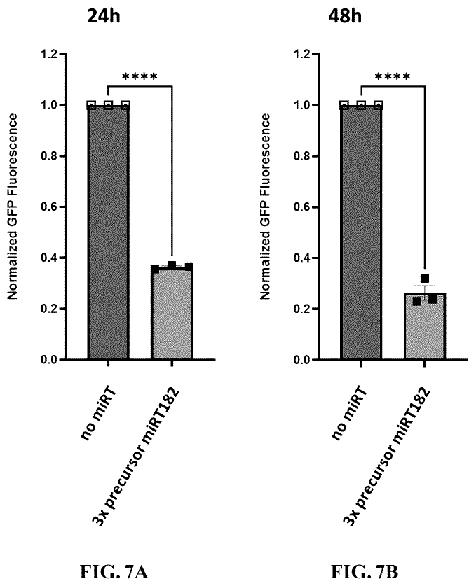

FIG. 7 shows the quantification of GFP fluorescence in transfected HEK293 cell lysate, and demonstrates that the presence of the precursor miR182 target site significantly inhibits GFP expression. Of note, the inhibition of GFP expression is maintained over time, with similar levels of inhibition observed 24 h after medium replacement (FIG. 7A) and 48h after medium replacement (FIG. 7B).

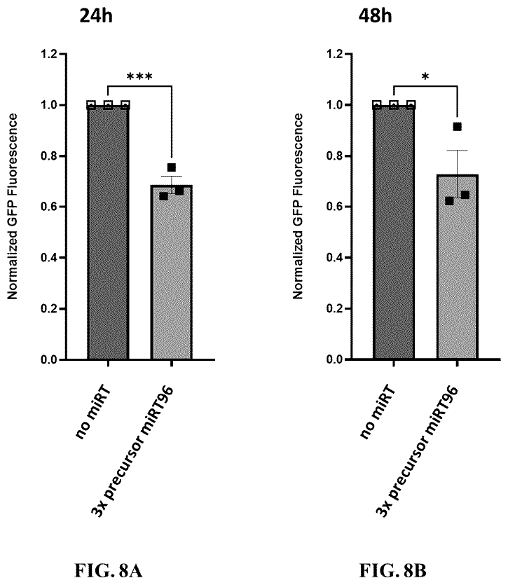

FIG. 8 shows the quantification of GFP fluorescence in transfected HEK293 cell lysate, and demonstrates that the presence of the precursor miR96 target site significantly inhibits GFP expression. Of note, the inhibition of GFP expression is maintained over time, with similar levels of inhibition observed 24 h after medium replacement (FIG. 8A) and 48h after medium replacement (FIG. 8B)

These data provide evidence that, similar to the so-called precursor miR183 target site (SEQ ID NO: 1), the so-called precursor miR182 target site (SEQ ID NO: 21) and the so-called precursor miR96 target site (SEQ ID NO: 24) are able to significantly inhibit the expression of a gene in cells expressing miR182 or miR96, respectively. The so-called precursor miR target sites of the miR183 family as described herein may thus be used, for example inserted in an expression cassette or a vector, to regulate the expression of a nucleic acid of interest, in particular to prevent the expression of the nucleic acid of interest in cells expressing miRNA of the miR183 family.

1-15. (canceled)

16. An isolated nucleic acid sequence comprising at least two copies of a miRNA target site of the miR183 family, wherein said miRNA target site has a sequence as set forth in SEQ ID NO: 1, SEQ ID NO: 21, or SEQ ID NO: 24, or a sequence having at least 90% identity with any one of SEQ ID NO: 1, SEQ ID NO: 21, or SEQ ID NO: 24.

17. The isolated nucleic acid sequence according to claim 16, wherein said miRNA target site of the miR183 family is a miR183 target site having a sequence as set forth in SEQ ID NO: 1 or a sequence having at least 90% identity with SEQ ID NO: 1.

18. The isolated nucleic acid sequence according to claim 16, wherein said isolated nucleic acid sequence comprises two to six copies of the miRNA target site of the miR183 family.

19. The isolated nucleic acid sequence according to claim 16, wherein said isolated nucleic acid sequence comprises three copies of the miRNA target site of the miR183 family.

20. The isolated nucleic acid sequence according to claim 16, wherein said isolated nucleic acid sequence comprises a sequence as set forth in SEQ ID NO: 7, SEQ ID NO: 23, or SEQ ID NO: 26, or a sequence having at least 90% identity with any one of SEQ ID NO: 7, SEQ ID NO: 23, or SEQ ID NO: 26.

21. The isolated nucleic acid sequence according to claim 16, wherein the copies of the miRNA target site of the miR183 family are separated by a spacer.

22. An expression cassette comprising a promoter, a gene of interest, and a regulatory element comprising at least one copy of a miRNA target site of the miR183 family, or a vector comprising said expression cassette, wherein said miRNA target site has a sequence as set forth in SEQ ID NO: 1, SEQ ID NO: 21, or SEQ ID NO: 24, or a sequence having at least 90% identity with any one of SEQ ID NO: 1, SEQ ID NO: 21, or SEQ ID NO: 24.

23. The expression cassette or vector according to claim 22, wherein said regulatory element comprises at least one copy of a miR183 target site having a sequence as set forth in SEQ ID NO: 1 or a sequence having at least 90% identity with SEQ ID NO: 1.

24. The expression cassette or vector according to claim 22, wherein said regulatory element comprises two to six copies of the miRNA target site of the miR183 family, the copies being optionally separated by a spacer.

25. The expression cassette or vector according to claim 22, wherein said regulatory element comprises three copies of the miRNA target site of the miR183 family, the copies being optionally separated by a spacer.

26. The expression cassette or vector according to claim 22, wherein said regulatory element comprises a sequence as set forth in SEQ ID NO: 7, SEQ ID NO: 23, or SEQ ID NO: 26, or a sequence having at least 90% identity with any one of SEQ ID NO: 7, SEQ ID NO: 23, or SEQ ID NO: 26.

27. The expression cassette or vector according to claim 22, wherein said regulatory element further comprises at least one copy of another miRNA target site.

28. The expression cassette or vector according to claim 22, wherein said regulatory element further comprises at least one copy of another miRNA target site of the miR183 family.

29. The expression cassette or vector according to claim 22, wherein said regulatory element is operably linked to or inserted in the gene of interest.

30. The expression cassette or vector according to claim 22, wherein said regulatory element is inserted in the 3′-UTR of the gene of interest.

31. A vector comprising a regulatory element comprising at least one copy of a miRNA target site of the miR183 family, wherein said miRNA target site has a sequence as set forth in SEQ ID NO: 1, SEQ ID NO: 21, or SEQ ID NO: 24, or a sequence having at least 90% identity with any one of SEQ ID NO: 1, SEQ ID NO: 21, or SEQ ID NO: 24.

32. The vector according to claim 31, wherein said regulatory element comprises a sequence as set forth in SEQ ID NO: 7, SEQ ID NO: 23, or SEQ ID NO: 26, or a sequence having at least 90% identity with any one of SEQ ID NO: 7, SEQ ID NO: 23, or SEQ ID NO: 26.

33. A pharmaceutical composition comprising the expression cassette or vector according to claim 22, and at least one pharmaceutically acceptable excipient.

34. A medicament comprising the expression cassette or vector according to claim 22.

35. A method for modulating or controlling the expression of a nucleic acid of interest in a tissue comprising at least two different cell types, said method comprising introducing into said tissue the expression cassette or vector according to claim 22.
